(19) United States
(12) Patent Application Publication
(10) Pub. No.: US 2007/0077649 A1
(43) Pub. Date: Apr. 5, 2007

(54) Transplantable cell growth niche and related compositions and methods

(76) Inventors: Paul J. Sammak, Pittsburgh, PA (US); Lauren E. Kokai, Pittsburgh, PA (US); Kacey G. Marra, Houston, PA (US)

Correspondence Address: JESSE A. HIRSHMAN, ESQ., 1722 MURRAY AVENUE, PITTSBURGH, PA, 15217, US

(21) Appl. No.: 11/516,076

(22) Filed: Sep. 6, 2006

Related U.S. Application Data
(60) Provisional application No. 60/714,731, filed on Sep. 6, 2005.

(51) Int. Cl. C12N 5/08; C12N 5/06

(52) U.S. Cl. 435/325; 435/368

(57) ABSTRACT

Provided is a method of culturing a successor cell from a precursor cell comprising co-culturing a precursor cell such as, without limitation, a human embryonic stem cell or a neuronal stem cell with an adult mesenchymal stem cell. In one embodiment, the adult mesenchymal stem cell is derived from adipose tissue. Also provided is a composition comprising mesenchymal stem cells in a growth matrix biologically-compatible with the cells. In another embodiment, a method of regenerating tissue is provided comprising introducing into a patient a cell growth niche comprising either or both of mesenchymal stem cells and neuronal stem cell or successor cell in a growth matrix biologically-compatible with the cells. In another embodiment, a method of regenerating tissue is provided comprising introducing into a patient a cell growth niche comprising matrix, cells and slow release polymer beads containing growth factors.

### CROSS REFERENCE TO RELATED APPLICATIONS

This application claims priority to U.S. Provisional Patent Application Nos. 60/714,731, filed on Sep. 6, 2005, and which is incorporated herein by reference in its entirety.

### STATEMENT REGARDING FEDERAL FUNDING

This invention was made with government support under Grant No. R37 HD012913-22S1, awarded by the National Institutes of Health. The government has certain rights in this invention.

### BACKGROUND

1. Field of the Invention

Methods of culturing neuronal stem cells and progeny are provided, as well as related compositions and products.

2. Description of the Related Art

Human embryonic stem cells (hESCs) hold dramatic promise for generating differentiated cells for a number of purposes, including cell replacement and transplantation therapies, as well as drug discovery and understanding the underlying basis for disease and, even more generally, organism growth. In one non-limiting example, ESCs may be useful in growing cells of neuronal lineages that can be used for treatment of amyotrophic lateral sclerosis (ALS) by replacing damaged neural stem cells (NSC), neurons, astrocytes or oligodendrocytes.

It was first documented in 1994 that pluripotent cells could be isolated from human blastocysts. Since then, techniques for deriving and culturing human ES cells have been refined and their promise in regenerative medicine has burgeoned. As pluripotent cells, human embryonic stem cells are able to form teratomas that contain derivatives of all three primary germ layers (mesoderm, endoderm, and ectoderm) when injected under the skin of immunosuppressed mice and are able to proliferate for long periods without losing their pluripotentcy. For these reasons, human ESCs are especially attractive as a source of replacement cells in regenerative therapy for destroyed or dysfunctional cells. This type of therapy would be most critical for neurological diseases where the original tissue can not always be repaired by the local stem cells to a functional state. See, e.g., Stem Cells: Scientific Progress and Future Research Directions. Department of Health and Human Services. June 2001. http://stemcells.nih.gov/info/scireport.

The in vitro growth characteristics of human ES cells differ from mouse ES cells because of their lack of response to Leukemia Inhibitory Factor. While LIF is able to maintain mouse ESCs in an undifferentiated state for long periods, culture of existing human cell lines has been dependent on co-culturing with mitotically inactivated primary mouse fibroblasts, which serve as feeder cells. Well-characterized mouse feeder layers include murine bone marrow-derived stromal feeder cell lines (MS5s or PA6s) or primary stromal feeder cells obtained from mouse embryonic fibroblasts (MEFs). In addition, hESCs are cultured with either fetal bovine serum or serum replacement in the presence of bFGF (basic Fibroblast Growth Factor). If the feeder cells are removed, the ESCs differentiate spontaneously and unpredictably.

One example of problems ESCs will present in clinical use is the possibility that transplantation of ESCs will introduce nonhuman pathogens or antigens into the host. For example, both animal-derived serum replacements and non-human feeder layers used in ESC cultures are sources of the nonhuman sialic acid Neu5Gc against which many humans have circulating antibodies. It has been shown by Martin et al. (Martin, M. J., et al., Human embryonic stem cells express an immunogenic nonhuman sialic acid. Nat Med, 2005. 11(2): p. 228-32) that Human Embryonic Stem Cells cultured with animal-derived serum replacements and/or non-human feeder layers incorporate Neu5Gc and, when in contact with human serum, elicit increased levels of IgG antibodies.

In an effort to make ESCs more clinically applicable, several groups have developed culture systems that partially or completely avoid the use of animal products. For example, human feeders of different tissue origin have been shown to successfully maintain undifferentiated ESCs, however certain ethical issues may prevent the large scale use of some of these culture systems.

An alternative to allogenic human feeders would be an autogeneic feeder layer, to further reduce the possibility of immune rejection. We and others including Xu et al (Stem Cells November 2004; 22(6), 972-980) and Stojkovic et al. (An autogeneic feeder cell system that efficiently supports growth of undifferentiated human embryonic stem cells. Stem Cells. March 2005; 23(3):306-14) have found that hESCs removed from mouse feeders could spontaneously differentiate to fibroblast like cells which could be isolated and replated to provide a feeder cell layer. Thus, pluripotent ESCs could be plated and maintained on fibroblast cells of human origin.

Moving away from feeder layers, several groups have found that by using conditioned media with modified surfaces it is possible to maintain a pluripotent ESC culture system. In one example, plates were coated with extracellular matrix proteins and cells were cultured in knockout DMEM conditioned by mitomycin-C treated MEFs. Alternatively, it was shown by Xu et al. (Noggin and bFGF cooperate to maintain the pluripotency of human embryonic stem cells in the absence of feeder layers. Nature Methods, March 2005 2(3); 185-90, noggin is used to counter contaminants in the knockout serum replacer), that the combination of bone morphogenetic protein antagonist noggin within the context of knockout serum replacers and basic fibroblast growth factor are critical for preventing hESC differentiation in absence of feeder cells. A fully defined cell culture media has been used by Ludwig et al., and has been used without animal products. However, it is cost prohibitive and current formulations still rely on animal proteins including Matrigel and bovine serum albumin. Nature Methods August 2006 3(8) 637-646.

Additional concerns have been raised regarding the transplantation of ESC populations. The first concerns the method in which ESCs are cultured. ESCs grow and differentiate in colonies. Should any cell within that colony not differentiate before transplantation, a teratoma could result even if it comprised only a small fraction of the transplant. Therefore, protocols must exist to completely differentiate ESC populations to the desired cell type before implantation can occur.

Additionally, undifferentiated human ESCs assayed by fluorescence activated cell sorting exhibit low expression of MHC-I. Even though the expression level increased eight to ten fold following differentiation, levels were still ten fold lower than those of other somatic cells.

Stem cells also are present in adults. An adult stem cell (somatic stem cell) is an undifferentiated cell found among differentiated cells in a tissue or organ. It can renew itself and can differentiate to yield the major specialized cell types of the tissue or organ. The primary roles of adult stem cells in a living organism are to maintain and repair the tissue in which they are found. Unlike embryonic stem cells, which are defined by their origin (the inner cell mass of the blastocyst), the origin of adult stem cells in mature tissues is unknown, but is thought to derive from primitive cells produced during fetal development.

Adult (not embryonic) stem cells have been found in many somatic tissues. Stem cells reside in specific tissues where they maintain tissue due to normal wear and tear. They may remain largely quiescent (non-dividing) for many years until they are activated in large numbers by disease or tissue injury. The adult tissues reported to contain stem cells include brain, bone marrow, peripheral blood, blood vessels, skeletal muscle, skin and liver and others. Certain kinds of adult stem cells have the ability to differentiate into a number of different cell types, given the right conditions. One population of adult stem cells, hematopoietic stem cells, forms all the types of blood cells in the body. A second population, bone marrow stromal cells (mesenchymal stem cells), are a mixed cell population that generates bone, cartilage, fat, and fibrous connective tissue. The adult brain also contains neural stem cells that are able to generate the brain's three major cell types—astrocytes and oligodendrocytes, which are non-neuronal cells, and neurons. Certain adult stem cells exhibit plasticity in that they can differentiate into tissue other than their tissue of origin.

Cell replacement in the central nervous system (CNS) remains a challenging goal because of the lack of safe and effective donor cells, as well as difficulty in remodeling the nonneurogenic adult CNS environment (Zhang, S. C., Embryonic stem cells for neural replacement therapy: prospects and challenges. J Hematother Stem Cell Res, 2003. 12(6): p. 625-34). Several laboratories have developed protocols for producing neuronal lineages from human embryonic stem cells (hESCs). This involves a two-stage approach to first develop NSCs followed by further differentiation with defined growth factors (Barberi, T., et al., Neural subtype specification of fertilization and nuclear transfer embryonic stem cells and application in parkinsonian mice. Nat Biotechnol, 2003. 21(10): p. 1200-7; Perrier, A. L., et al., Derivation of midbrain dopamine neurons from human embryonic stem cells. Proc Natl Acad Sci U S A, 2004. 101 (34): p. 12543-8; and Murashov, A. K., et al., Directed differentiation of embryonic stem cells into dorsal interneurons. FASEB J. February 2005;19(2):252-4. Epub Nov. 15, 2004; Li, X.-J., et al., Specification of motoneurons from human embryonic stem cells. Nat Biotechnol. February 2005; 23(2):215-21. Epub Jan. 30, 2005). NSCs can be produced from embryoid bodies (Li, X.-J., et al., Specification of motoneurons from human embryonic stem cells. Nat Biotechnol. February 2005; 23(2):215-21. Epub Jan. 30, 2005) or by culture on preadipocytic stem cell lines (MS5 and PA6) derived from mouse bone marrow (Barberi, T., et al., Nat Biotechnol, 2003. 21(10): p. 1200-7 and Perrier, A. L., et al., Proc Natl Acad Sci U S A, 2004. 101(34): p.12543-8). Production of NSCs on a mesenchymal stromal cell feeder layer has several advantages including positive developmental signals that eliminate teratoma-forming pluripotent cells, dramatic proliferative amplification of stable multipotent NSC colonies. Evidence suggests that transplanting stromal cells along with neural cells improves both short and long term survival by a paracrine effect (Aggarwal, S. and M. F. Pittenger, Human mesenchymal stem cells modulate allogeneic immune cell responses. Blood, 2005. 105(4): p. 1815-22; Di Nicola, M., et al., Human bone marrow stromal cells suppress T-lymphocyte proliferation induced by cellular or nonspecific mitogenic stimuli. Blood, 2002. 99(10): p.3838-43; and Tse, W. T., et al., Suppression of allogeneic T-cell proliferation by human marrow stromal cells: implications in transplantation. Transplantation, 2003. 75(3): p.389-97). Stromal feeder lines such as MS5 and PA6 preadipocyte bone stromal cells have become a standard for the directed differentiation of embryonic stem cells to neural precursors and specific neural subtypes (Barberi, T., et al., Neural subtype specification of fertilization and nuclear transfer embryonic stem cells and application in parkinsonian mice. Nat Biotechnol, 2003. 21(10): p. 1200-7).

NSCs derived from hESCs can be further differentiated into several neuronal lineages including dopaminergic, peripheral and sensory neurons (Perrier, A. L., et al., Derivation of midbrain dopamine neurons from human embryonic stem cells. Proc Natl Acad Sci U S A, 2004. 101(34): p. 12543-8; Pomp, O., et al., Generation of peripheral sensory and sympathetic neurons and neural crest cells from human embryonic stem cells. Stem Cells, 2005: p. 2005-0038; and Yamazoe, H., et al., Collection of neural inducing factors from PA6 cells using heparin solution and their immobilization on plastic culture dishes for the induction of neurons from embryonic stem cells. Biomaterials, 2005. 26(28): p. 5746-54). Astrocytes, radial glia and oligodendrocytes have also been derived from hESC ((Perrier, A. L., et al., Derivation of midbrain dopamine neurons from human embryonic stem cells. Proc Natl Acad Sci U S A, 2004. 101(34): p. 12543-8; Zhang, S. C., et al., In vitro differentiation of transplantable neural precursors from human embryonic stem cells. Nat Biotechnol, 2001. 19(12): p. 1129-33; Liour, S. S. and R. K. Yu, Differentiation of radial glia-like cells from embryonic stem cells. Glia, 2003. 42(2): p. 109-17; and Nistor, G. I., et al., Human embryonic stem cells differentiate into oligodendrocytes in high purity and myelinate after spinal cord transplantation. Glia, 2005. 49(3): p. 385-96). A disadvantage of the NSC production on mouse stromal cells is the potential for transfer of animal specific sialic acid Neu5Gc (Martin, M. J., et al., Human embryonic stem cells express an immunogenic nonhuman sialic acid. Nat Med, 2005. 11(2): p. 228-32). Propagation of pluripotent cells on human fibroblast feeder layers has been accomplished and would avoid xeno-transplant incompatibility (Richards M, F. C., Chan W K, Wong P C and Ariff Bongso, Human Feeders Support Prolonged Undifferentiated Growth of Human Inner Cell Masses and Embryonic Stem Cells. Nature Biotechnology, 2002. 20: p. 933-936; Miyamoto K, H. K., Suzuki T, Ichihara S, Tamada T, Kano Y, Yamabe T, and Yoshihiro Ito, Human Placenta Feeder Layers Support Undifferentiated Growth of Primate Embryonic Stem Cells. Stem Cells, 2004. 22: p. 433-440; Choo A B H, P. J., Chin A C P, Oh S K W, Expansion of Pluripotent Human Embryonic Stem Cells on Human Feeders. Biotechnology and Bioengineering, 2004. 88(3): p. 321-331; Hovatta O, M. M., Gertow K, Stromberg A M, Inzunza J, Hreinsson J, Rozell B, Blannow E, Andang M, and Lars Ahrlund-Richter, A Culture System Using Human Fibroblasts as Feeder Cells Allows Production of Human Embryonic Stem Cells. European Society of Human Reproduction and Embryology, 2003. 18(7): p. 1404-1409; Inzunza J, G. K., Stromberg M A, Matilainen E, Blennow E, Skottman H, Wolbank S, Ahrlund-Richter L, and Outi Hovatta, Derivation of Human Embryonic Stem Cell Lines in Serum Replacement Medium Using Postnatal Human Fibroblasts as Feeder Cells. Stem Cells, 2005. 23: p. 544-549; Amit M, M. V., Segev H, Shariki K, Laevsky I, Coleman R, and J. Itskovitz-Eldor, Human Feeder Layers for Human Embryonic Stem Cells. Biology of Reproduction, 2003. 68: p. 2150-2156; and Cheng L, H. H., Ye Z, Zhan X, and Guatam Dravid, Human Adult Marrow Cells Support Prolonged Expansion of Human Embryonic Stem Cells in Culture. Stem Cells, 2003. 21: p. 131-142). However, human feeder cells have been evaluated only for maintaining, but not differentiating hESC.

### SUMMARY

In one embodiment of the methods disclosed herein, a method of culturing a successor cell from a precursor cell is provided. The method comprises co-culturing a precursor cell, such as, without limitation a human embryonic stem cell or a human neuronal stem cell, with an adult mesenchymal stem cell. In one embodiment, the adult mesenchymal stem cell is derived from adipose tissue. In another embodiment, the successor cell is one of an astrocyte, an oligodendrocyte and a neuron. In yet another embodiment, the method further comprises co-culturing the precursor cell and the adult mesenchymal stem cell on a growth matrix biologically-compatible with the cells. The growth matrix can comprise collagen and/or a polymer, such as a hydrogel, for example and without limitation, one or more of polyesters, polysaccharides, poly(lactic acid), poly(glycolic acid), poly(lactic-co-glycolic acid) copolymers, poly(vinyl alcohol), polycaprolactone, polyethyleneglycol and gelatin. The growth matrix also can comprise, without limitation, one or more of a growth factor, a differentiation factor, an adhesion factor and a migration factor, such as, without limitation, laminin, glial derived neurotrophic factor, cilliary neurotrophic factor and fibroblast growth factor 2 or a polypeptide comprising one or more of the following amino acid sequences: RGD, YIGSR (SEQ ID NO: 1), IKVAV (SEQ ID NO: 2) and GRGDS (SEQ ID NO: 3). In one embodiment, the precursor cell and the adult mesenchymal stem cell are obtained from the same patient, such as a human patient. In another embodiment, the adult mesenchymal stem cells are obtained from a same human patient (autologous cells) and the precursor cells are hESCs. As shown below, the density of the support cells, such as the adult mesenchymal stem cell, may be modulated to determine successor cell type.

In another embodiment disclosed herein, a composition is provided comprising mesenchymal stem cells in a growth matrix biologically-compatible with the cells. Various embodiments of the growth matrix are described above and throughout this document.

In yet another embodiment, a method of regenerating tissue comprising introducing into a patient a cell growth niche. The niche comprises mesenchymal stem cells in a growth matrix biologically-compatible with the cells. The growth matrix is as described above and throughout this document. In one embodiment, the mesenchymal stem cells are human. In another embodiment, the mesenchymal stem cells are obtained from the patient. In yet another embodiment, the mesenchymal stem cells are derived from adipose tissue.

Lastly, a method of repairing damage to a patient's nervous system is provided, comprising transplanting into the patient's nervous system a composition comprising mesenchymal stem cells in a growth matrix biologically-compatible with the cells. Various embodiments of the growth matrix are described above and throughout this document. In one embodiment, the growth matrix comprises one or more of a growth factor, a differentiation factor and a migration factor, such as, without limitation, a neurotropic factor, such as, without limitation one or more of glial derived neurotrophic factor, cilliary neurotrophic factor and fibroblast growth factor 2. In another embodiment, the growth matrix comprises one or both of collagen and a hydrogel. In one embodiment, the patient has ALS.

### BRIEF DESCRIPTION OF THE DRAWINGS

FIG. 1 shows pluripotent Markers in hESC lines H1 (A, B, D) and HSF-6 (C, E, F). Panels A and B show Oct-4 immunostaining of pluripotent HSF-6 colony at 10× (A) and 40× (B). Individual nuclei are clearly labeled in (B). Note the metaphase plate at right center of the stem cell colony. Panel C shows Oct-4 mRNA is expressed in pluripotent cell colonies (lane 4) but is lost soon after differentiation begins (lane 2) as evidenced by RT-PCR analysis. Differentiating cells were identified morphologically and selectively scraped from the perimeter of one colony. Panel D shows SSEA-3 immunostaining of most cells in one colony. Panel E shows SSEA-4 immunostaining is generally more variable in colonies compared to SSEA-3, while panel F shows SSEA-1 is always negative in hESC.

FIG. 2 are photomicrographs showing differentiation of hESC line HSF6 to neuronal lineages with low density feeder cells. HSF6 were normally maintained on mouse embryonic fibroblasts (CF-1) cells at 120,000 to 150, 000 cells per well in 6 well dishes, but by reducing the density of feeder cells to 50,000 cells/well, directed differentiation takes place with over 80% of cells initially staining with the neural stem cell marker, nestin. At 2-3 weeks, neuron specific markers, Mushashi-1, NCAM and beta III tubulin predominate, with little staining of astrocytes (GFAP) or oligodendrocytes (Olig-1 or A2B5, not shown).

FIG. 3 are photmicrographs showing cell death at injection site, pluripotent cell survival in ventricles 24 hrs after injection of undifferentiated hESCs. Panel (a) shows Hematoxylin and eosin (H&E) stained sections of mouse brain injected 1×10⁶ undifferentiated hESCs showing extensive nuclear fragmentation and cell death. Panel (b) shows that as early as 24 hrs after injection hESCs were found lining the ventricles. HESC were pre-labeled with CMFDA (green) and CM-DiI (red) and immunostained for Oct-4 (blue). Bar in a, 20 μm, b; 100 μm.

FIG. 4 are photomicrographs showing that injected NSCs move to distant sites at ventricles and at co-injected tumor cells H&E stained mouse brain injected with U87 glioblastoma tumor cells (upper square) and 1×10⁶ NSCs derived from hESCs (lower rectangle to right) at day 5. Far left of the lower box shows a stream of NSCs extending to in the ventricle. Cells labeled before injection with CMFDA (green) or DiI (red) are seen in the ventricle (2E) and at the perimeter of the tumor mass (2F). Bar in (a), 1000 μm; (b) 20 μm.

FIG. 5 provides photomicrographs showing greater differentiation of hESC into neurons on patient-specific hASCs than on MS5 preadipocytes. Mitomycin treated mouse MS5 bone stromal preadipocytes (panel a) and hASCs from a 60 yr old patient (panel b) were plated at 100,000 cells per well. The following day, hESCs were passaged at 1:2 and plated in colonies of 50-100 cells. Cells were grown in DMEM containing 15% knockout serum replacement, non-essential amino acids and 2-mercaptoethanol for 30 days, and then switched to N2 media for 30 days. Neurite networks (green-β-III tubulin) on feeders (blue-Hoechst DNA stain) extended from hESC-derived neuronal colonies hASCs supported a dramatically greater number of neurons and more complex neurite networks than did mouse bone stromal cells at similar densities. Repeated 3×. Bar in b, 20 μm.

FIG. 6 provides photomicrographs showing hESC differentiation into neurons and oligodendrocytes or into astrocytes. Mitomycin treated hASCs from a 38 yr old patient were plated at 150K (panels a and b), 50K (panels c and d) cells per well in a 6-well plate. After 24 hours, hESCs were passaged at 1:10 and plated in colonies of 50-100 cells and grown for an additional 19 days. Panels (a) and (b) show, III tubulin (neurons, green) and A2B5 (oligodendrocytes, red) at 150 ASC/well. Panels (c) and (d) show nestin (neural stem cell, green) and GFAP (astrocyte, red) at 50 ASC/well. At high density ASCs, Oligodendrocyte and Neuronal staining co-localize in a subset of neurons (yellow and red in panel (a)) suggesting coordination of the two cell types (myelination or co-expression). At low density ASCs, astrocyte populations predominate with some nestin staining both with and without GFAP staining suggesting that either astroglia or other immature progenitor cells such as radial glia are mixed with astrocytes. Bars in panels (a) and (c) are 100 μm and in panels (b) and (d) are 20 μm.

FIG. 7 is a graph showing that nestin expression depends on hASC density. Nestin (an NSC marker) was detected by confocal microscopy of 3 colonies in each of 3 independent wells. Quantification of single optical sections was performed on the Leica TCS SP2 software. Nestin was statistically higher at 100 k and 150 k hASC than at 50 k hASC (P >0.001 and 0.005, respectively, 5 measurements on 6 colonies).

FIG. 8 is a graph showing controlled release of FGF-2 encapsulated within PLGA microspheres (in vitro). The in vitro release of FGF-2 from poly(D,L-lactic acid-co-glycolic acid) [PLGA] microspheres in PBS was examined over 14 days and was quantified by ELISA. A burst release was observed in the first 24 hours. After 3 days, the release had reached steady-state, and most of the FGF-2. had been released by day 14.

FIG. 9 illustrates that Cultisphers support hASC incorporation and transplanted hASCs beads support neuron growth in vivo. Panel (a) is a SEM of a cut Cutispher 200 μm diameter (image from Percell Biolytica, Å storp, Sweden) showing 20 μm pores that support and protect cells. Panel (b) shows Cultisphers seeded with hASCs stained with Dapi. Panel (c) shows survival of hASCs in Cultisphers within a polycaprolactone nerve guide transplanted into Rat sciatic nerve, is shown in section with immunostaining for human specific nuclear lamin (yellow). In panel (d), a nerve guide containing Cultisphers seeded with hASCs and implanted in a rat sciatic nerve defect was stained for live cell membrane marker PKH26 (red) showing transplanted hASC, dapi (blue) and neurofilament protein (green) showing infiltration of endogenous rat neurons. In the absence of hASCs, neurofilament staining was not seen (not shown).

FIGS. 10A and 10B show ASC FACS Analysis for the marker CD146 (mel-CAM, Muc-18). FIG. 10A is a control.

FIGS. 11A and 11B show ASC FACS Analysis for the markers CD34 and CD90 (Thy-1), respectively.

FIG. 12 shows ASC FACS Analysis for the marker CD49d.

FIGS. 13A and 13B are photomicrographs showing expression of Oct4 in 19 day co-cultured human fibroblasts-hESC (FIG. 13A) and ASC-hESC (FIG. 13B).

FIGS. 14A and 14B are photomicrographs showing expression of Pax6 in 19 day co-cultured fibroblasts-hESC (FIG. 14A) and ASC-hESC (FIG. 14B).

FIGS. 15A-15C are photomicrographs showing expression of Nestin in hESCs co-cultured for 19 days with ASCs. ASC density was: 50,000 ASCs per 10 cm² well (FIG. 15A), 100,000 ASCs per 10 cm² well (FIG. 15B) and 150,000 ASCs per cm² well (FIG. 15C).

FIG. 16 is a graph showing Nestin protein expression by HSF-6 ESCs co-cultured for 19 days with ASCs.

FIGS. 17A-17C are photomicrographs showing expression of βIII-tubulin hESCs co-cultured for 19 days with ASCs. ASC density was: 50,000 ASCs per cm² well (FIG. 17A), 100,000 ASCs per cm² well (FIG. 17B) and 150,000 ASCs per cm² well (FIG. 17C).

FIG. 18 is a graph showing III-tubulin protein expression by HSF-6 ESCs co-cultured for 19 days with ASCs.

FIGS. 19A-19C are photomicrographs showing expression of A2B5 in hESCs co-cultured for 19 days with ASCs. ASC density was: 50,000 ASCs per cm² well (FIG. 19A), 100,000 ASCs per cm² well (FIG. 19B) and 150,000 ASCs per cm² well (FIG. 19C).

FIG. 20 is a graph showing A2B5 protein expression by HSF-6 ESCs co-cultured for 19 days with ASCs.

FIGS. 21A-21C are photomicrographs showing expression of Glial Fibrillar Associated Protein (GFAP) in hESCs co-cultured for 19 days with ASCs. ASC density was: 50,000 ASCs per cm well (FIG. 21A), 100,000 ASCs per cm² well (FIG. 21B) and 150,000 ASCs per cm² well (FIG. 21C).

FIG. 22 is a graph showing GFAP protein expression by HSF-6 ESCs co-cultured for 19 days with ASCs.

FIGS. 23A-23D are photomicrographs showing expression of βIII-tubulin in differentiated hESC co-cultured with ASCs for 60 days. FIGS. 23A and 23C are 10× views and FIGS. 23B and 23D are 20× views of FIGS. 23A and 23C, respectively.

### DETAILED DESCRIPTION

Disclosed herein are methods of producing differentiated cell populations from either embryonic stem cells or adult stem cells. In one embodiment, human embryonic stem cells or adult stem cells (precursor cells) are co-cultured with human mesenchymal stem cells or other more differentiated human support cells to produce successor cells, such as, without limitation and in the context of neuronal development, neuronal stem cells, neurons, astrocytes and oligodendrocytes. In another embodiment, a niche, and in a further embodiment, a transplantable niche, is provided to serve as a scaffold for differentiation of precursor cells into successor cells. The transplantable niche comprises a substrate or growth matrix comprising appropriate support cells, such as, without limitation, mesenchymal stem cells (for example and without limitation, human adipose-derived stem cells (hASC) or bone marrow stromal cells) or differentiated support cells (for example and without limitation, fibroblasts or other mesodermal support cells and astrocytes).

As used herein, in the context of cell lineage and cell differentiation, a precursor cell is a less-differentiated form of a related successor cell. Thus, in the context of neuronal cells, an embryonic stem cell is a precursor cell to both neural stem calls and neurons, astrocytes and oligodendrocytes. A neuronal stem cell is a successor to an embryonic stem cell, but a precursor to neurons, astrocytes and oligodendrocytes. As follows, neurons, astrocytes and oligodendrocytes are successors to embryonic stem cells and neural stem cells.

A growth matrix is a substrate upon which cells can grow. In the context of the present disclosure, a growth matrix must be bio-compatible with the cells grown therein, as well as, when relevant, with an organism into which a matrix is transplanted according to one embodiment of the present disclosure. By bio-compatible it is meant that the growth matrix does not substantially affect growth of desired cell populations within or upon the matrix, and does not produce any substantial deleterious effects when transplanted into an organism (from a statistical standpoint in a population).

A growth matrix may comprise any bio-compatible substance that can support growth of cells. In one embodiment, a growth matrix is a porous material. A growth matrix may have any useful/desirable shape, such as spherical, and may be engineered to any desired shape that is useful, such as a tubular or cylindrical shape, as a sheet or in the shape of a defect to be corrected. In one embodiment, the growth matrix comprises collagen. In another embodiment, the growth matrix comprises a hydrogel. In yet another embodiment, the matrix is produced from an biodegradable polymer so that the matrix dissolves over time, for example and without limitation, in the context of a transplantable niche, over a time period sufficient to permit sufficient infiltration of the matrix with cells and growth and differentiation of cells within the matrix. Non-limiting examples of suitable polymers include: polyesters, polysaccharides, poly(lactic acid), poly(glycolic acid), poly(lactic-co-glycolic acid) copolymers, poly(vinyl alcohol), polycaprolactone, polyethyleneglycol and gelatin. Depending on the use of the growth matrix, one or more of the polymers may be preferred over another.

One non-limiting example of a growth matrix is a collagen matrix, such as, without limitation, a Cultispher. Polymer matrices may be associated with (incorporated into, absorbed, adsorbed, covalently-linked or otherwise affixed to or within) a collagen matrix, as is known in the art (see, for example, Waddell, R. L., et al., Using PC12 cells to evaluate poly(caprolactone) and collagenous microcarriers for applications in nerve guide fabrication. Biotechnol Prog, 2003. 19(6): p. 1767-74; Bender, M. D., et al., Multi-channeled biodegradable polymer/CultiSpher composite nerve guides. Biomaterials, 2004. 25(7-8): p. 1269-1278). Certain substances that facilitate growth, differentiation, adhesion and migration of cells (for example and without limitation, a growth factor, a differentiation factor, an adhesion factor and a migration factor) may be associated with the growth matrix (See, for example, Meese, T. M., et al., Surface studies of coated polymer microspheres and protein release from tissue-engineered scaffolds. J Biomater Sci Polym Ed, 2002.13(2): p. 141-51; Hu, Y., J. O. Hollinger, and K. G. Marra, Controlled release from coated polymer microparticles embedded in tissue-engineered scaffolds. J Drug Target, 2001. 9(6): p. 431-8; Royce, S. M., M. Askari, and K. G. Marra, Incorporation of polymer microspheres within fibrin scaffolds for the controlled delivery of FGF-1. J Biomater Sci Polym Ed, 2004. 15(10): p. 1327-36 and DeFail, A. J. 2006, et al., Controlled Release of Bioactive TGF-β1 from Microspheres Embedded within Biodegradable Hydrogels. Biomaterials, 27(8):1579-85). Non-limiting examples of such factors, useful in neuronal growth include: genipin; laminin; a laminin peptide (e.g., RGD, YIGSR (SEQ ID NO: 1), IKVAV (SEQ ID NO: 2) and GRGDS (SEQ ID NO: 3)—see, e.g., Santiago, et al. 2006 “Peptide-Surface Modification of poly(caprolactone) with Laminin-Derived Sequences for Adipose-Derived Stem Cell Applications,” Biomaterials, 27:2962-9 and glial derived neurotrophic factor (GDNF), cilliary neurotrophic factor (CNTF) and fibroblast growth factor 2 (FGF-2). Growth matrices can be prepared by any suitable method, including, without limitation: photolithography, microfluidic patterning, electrochemical deposition, reactive ion etching, three-dimensional printing and photochemistry combined with laser technology (See, for example, Musoke-Zawedde et al. 2006 “Anisotropic Three-Dimensional Peptide Channels guide Neurite Outgrowth Within a Biodegradable Hydrogel Matrix, Biomed. Mater. 1:162-169).

Substances that facilitate growth, differentiation, adhesion and migration of cells (for example and without limitation, a growth factor, a differentiation factor, an adhesion factor and a migration factor) are associated with the growth matrix by any of a number of methods, including, without limitation, covalent linkage, absorption and adsorption. The substances can be incorporated within a delivery matrix that is optionally biodegradable, or from which the substance can diffuse or otherwise be distributed, such as, without limitation, a hydrogel bead or another polymer bead, as described herein and as are broadly known in the art.

In one specific embodiment, it surprisingly has been found that long-term support of hESC-derived neuronal lineages is provided by adult mesenchymal stem cells isolated from adipose tissue. Patient-specific human adipose-derived stem cells (hASC) provide a niche that directs differentiation to all neuronal lineages, depending on density. Support matrices, for example and without limitation, porous collagen beads can be employed to protect internalized hESC-derived neuronal lineages and hASC support cells from mechanical trauma during transplantation. Microspheres that slowly release growth factors can be incorporated into the matrix and allow for selective chemical modification of the stem cell niche.

As described in further detail below, preliminary experiments in each case were promising. Nevertheless, components must be optimized in cell culture before embryonic-adult stem cell niches can be transplanted into animal models. In addition, there were several obstacles to the eventual use of implantable stem cell niches in humans, including: 1) Controlled differentiation, outgrowth and integration of NSC and motor neurons, making sure to exclude potential cancer forming pluripotent cells; 2) Low cell viability due to immune rejection of non-human antigens from animal products; 3) Injury during transplantation and inability to recapitulate the endogenous 3-dimensional organization.

To address these issues, also described herein are stem cell niches, for example and without limitation, FDA-approved Cultisphers, which provide bioengineered delivery systems prepared from 3-dimensional matrices that are compatible with support cells, such as, without limitation, hASCs. These collagen-based matrices improve long-term viability, immunocompatibility and secretion of factors from neuroprotective cells such as dopamine-secreting retinal pigment epithelium in the brain in parkisonian animal models and in patients with neurodegenerative disease. Watts et al., 2003. J Neural Transm Suppl 65; 215-27. Within these matrices, one can optimize a combination of progenitor cells (for example and without limitation embryonic or neuronal stem cells), differentiation support cells (for example and without limitation, hASCs and astrocytes) and growth factors, differentiation factors, cell adhesion factors and/or migration factors designed to protect and facilitate growth and differentiation of cells within the matrix, for example and without limitation, motor neurons in an animal model of ALS from neurodegeneration without generating an extensive immune response.

A method of repairing damage to a patient's nervous system is provided according to one embodiment described herein, comprising transplanting into the patient's nervous system a composition comprising mesenchymal stem cells in a growth matrix biologically-compatible with the cells. Various embodiments of the growth matrix are described above and throughout this document. In one embodiment, the growth matrix comprises one or more of a growth factor, a differentiation factor and a migration factor, such as, without limitation, a neurotropic factor, such as, without limitation one or more of glial derived neurotrophic factor, cilliary neurotrophic factor and fibroblast growth factor 2. In another embodiment, the growth matrix comprises one or both of collagen and a hydrogel. In one embodiment, the patient has ALS.

Amyotrophic Lateral Sclerosis (ALS), which is characterized by muscle atrophy and motor neuron loss, has been modeled with mutations in the Cu—Zn superoxide dismutase (SOD1) in rats. Before motor neuron degeneration occurs, the gain of function mutation first produces astroglial defects, including loss of the astrocyte glutamate transporter EAAT2 and sensitivity to oxidative damage and glutamate excitotoxicity (Howland, D. S., et al., Focal loss of the glutamate transporter EAAT2 in a transgenic rat model of SOD1 mutant-mediated amyotrophic lateral sclerosis (ALS). Proc Natl Acad Sci U S A, 2002. 99(3): p. 1604-9 and Dunlop, J., et al., Impaired Spinal Cord Glutamate Transport Capacity and Reduced Sensitivity to Riluzole in a Transgenic Superoxide Dismutase Mutant Rat Model of Amyotrophic Lateral Sclerosis. J. Neurosci., 2003. 23(5): p.1688-1696). Further, SOD1 defects in chimeric mice can be ameliorated by wild type non-neuronal cells that are neuroprotective (Clement, A. M., et al., Wild-type nonneuronal cells extend survival of SOD 1 mutant motor neurons in ALS mice. Science, 2003. 302(5642): p. 113-7). Significantly transgenic human astrocytes modified using lentivirus to secrete glial derived neurotrophic factor (GDNF) transplanted into the lumbar spinal cord of rats overexpressing the G93A SOD1 mutation are locally neuroprotective (Klein, S. M., et al., GDNF delivery using human neural progenitor cells in a rat model of ALS. Hum Gene Ther, 2005. 16(4): p. 509-21). These studies and several others suggest that modifying the local microenvironment by cellular therapy may be a useful strategy for neuroprotection to extend the lives of ALS patients.

Treatment of patients directly with growth factors has had limited success because protein stability and delivery are problematic. One strategy to circumvent these problems has been to transplant supporting cells that provide a steady source of growth factors. Long-term conditioning of the local microenvironment could be obtained by growth factors secreted by transplanted cells including NSCs (Llado, J., et al., Neural stem cells protect against glutamate-induced excitotoxicity and promote survival of injured motor neurons through the secretion of neurotrophic factors. Mol Cell Neurosci, 2004.27(3): p. 322-31), astrocytes (Klein, S. M., et al., GDNF delivery using human neural progenitor cells in a rat model of ALS. Hum Gene Ther, 2005. 16(4): p. 509-21) and other stem cells. For example, mesenchymal stem cells (MSCs) from bone are neuroprotective when transplanted in the cortex and spinal cord after ischemic stroke by improving neurite outgrowth and motor control (Andrews, Abstract 104, ISSCR, 2005). Adult astrocytes from hippocampus, but not the spinal cord, are capable of regulating neurogenesis in vivo by instructing the stem cells to adopt a neuronal fate and by promoting proliferation (Song, H., C. F. Stevens, and F. H. Gage, Astroglia induce neurogenesis from adult neural stem cells. Nature, 2002. 417(6884): p. 39-44). This role in fate specification was unexpected because, during development, neurons are generated before most of the astrocytes. In addition, after spinal cord injury, endogenous subpial astrocytes can generate radial glial cells that contribute to neural stem cell populations and contribute to regeneration (Sei Shibuya, O.M.T.I.S.M.H.N., Temporal progressive antigen expression in radial glia after contusive spinal cord injury in adult rats. Glia, 2003. 42(2): p. 172-183). Therefore, certain astrocyte populations in combination with neural stem cells are important early contributors to repair and regeneration of the spinal cord.

In one non-limiting embodiment, in the context of ALS therapy, a goal would be to: 1) develop humanized differentiation protocols for hESC-derived NSCs, astrocytes and motor neurons to reduce graft rejection caused by animal proteins contaminating cell culture; 2) test implantable, 3-dimensional collagen Cultisphers that contain supportive hASC-hESC progeny for neuronal survival, differentiation and neurite outgrowth; 3) control delivery of encapsulated slow-release growth factors, GDNF, CNTF and FGF-2 to fine tune differentiation and integration of transplanted cells and to promote survival of motor neurons; 4) evaluate the combination of matrices containing astrocytes, NSCs, hASCs and growth factors in cell culture for conditions that promote hESC-derived motor neuron survival and electrophysiological function; 5) optimize transplanted cells and growth factors within matrices in wild type rats for viability of the transplants; and 6) evaluate these matrices in the ALS SOD1 rat model for support of degenerating motor neurons by measuring immunohistochemical markers for motor neurons and stem cell derivatives.

The present invention finds use in the following fields: tissue engineering; biological discovery; organ culture; derivation of tissue specific cells and cellular complexes; treatment of injury or degenerative disease through tissue replacement; and drug testing. Features of embodiments of the present invention include, without limitation: co-culture of adult stem cells to direct differentiation of embryonic stem cells to selected tissue and cellular fates; culture of stem cells and stem cell products in biomatrices to form a complex of cells, insoluble proteins and fibers, and soluble bioactive molecules and growth factors; complexes of cells matrices and bioactive molecules to form a stem cell niche that supports formation of stem cells or progenitor cells; transplantable stem cell niches contained within a matrix that can direct tissue repair or regeneration after disease or injury; and transplantable stem cell niches that limit host vs. graft disease and transplant rejection by physically containing allogenic cells to limit exposure to the immune system and the inclusion of immunosuppressive drugs or cells such as mesenchymal stem cells.

As stated above, a well characterized line of cells often used as embryonic feeder cells is the MS5 cell line which comprises murine bone marrow derived preadipocytic stromal cells. There are strong similarities between the MS5 cell line and Adipose Stem Cells. Adipose tissue is an abundant source of both preadipocytes and adipose stem cells, and therefore may be useful as a human feeder layer for human ESCs.

In vitro, ASCs do not provoke alloreactivity of incompatible lymphocytes and suppress mixed lymphocyte reaction and lymphocyte proliferative response to mitogens. In addition to the humanization of current ESC culture conditions, ASCs co-transplanted with hESCs may provide an immunosuppressive role that increases the ability of ESC to integrate into damaged tissue. Puissant et al. demonstrated that ASCs cocultured with mismatched HLA-DRB peripheral blood mononuclear cells did not ellicit PBMC proliferation and when cultured with phytohaemagglutin, ASCs suppressed PBMC reactivity (Puissant et al., BJH 129, 118-129 (2005)).

Presented herein is new evidence that human adipose derived mesenchymal stem cells (hASC) induce neural differentiation of hESC similarly to mouse MS5 cells. Adipose tissue, like bone marrow derived from the embryonic mesoderm, contains a heterogeneous stromal mesenchymal stem cell (MSC) population that has many advantages as a stem cell tissue source (Kang, S., et al., Improvement of Neurological Deficits by Intracerebral Transplantation of Human Adipose-Derived Stromal Cells After Cerebral Ischemia in Rats. Experimental Neurology, 2003.183: p. 355-366; Keyoung, H. M., et al., High-yield selection and extraction of two promoter-defined phenotypes of neural stem cells from the fetal human brain. Nat Biotechnol, 2001. 19(9): p. 843-50; Tchkonia, T., et al., Fat Depot Origin Affects Adipogenesis in Primary Cultured and Cloned Human Preadipocytes. Am. J. Physiol. Regul. Integr. Comp. Physiol, 2002. 282(5): p. R1286-1296; Maslowska, H., L. MacLean, and K. Cianflone, Regional Differences in Triacylglycerol Synthesis in Adipose Tissue and in Cultured Preadipocytes. Journal of Lipid Research, 1993.34: p. 219-228; Niesler, C., K. Siddle, and J. Prins, Human Preadipocytes Display a Depot-Specific Susceptibility to Apoptosis. Diabetes, 1998. 47(8): p. 1365-1368; Boney, C., et al., The Critical Role of Shc in insulin-like Growth Factor-I-Mediated mitogenesis and Differentiation in 3T3-L1 Preadipocytes. Mol. Endocrinol, 2000. 14(6): p. 805-813; Hausman, G. and R. Richardson, Newly Recruited and Pre-existing Preadipocytes in Cultures of Porcine Stromal-Vascular Cells:Morphology, Expression of Extracellular Matrix Components, and Lipid Accretion. J. Anim. Sci, 1998. 76(1): p. 48-60; Urso, B., et al., Comparison of Anti-Apoptotic Signalling by the Insulin Receptor and IGF-I Receptor in Preadipocytes and Adipocytes. Cell Signaling, 2001.13(4): p. 279-285; and Wabitsch, M., et al., IGF-I and IGFBP-3-Expression in Cultures Human Preadipocytes and Adipocytes. Horm Metab Res., 2000.32(11-12): p. 555-559). Both hASCs (a type of MSC) and bone marrow MSCs are capable of differentiating into similar lineages including chondrocytes, osteoblasts and myocytes (Keyoung, H. M., et al., High-yield selection and extraction of two promoter-defined phenotypes of neural stem cells from the fetal human brain. Nat Biotechnol, 2001. 19(9): p. 843-50; Sanchez-Ramos, J., et al., Adult Bone Marrow Stromal Cells Differentiate into Neural Cells in vitro. Experimental Neurology, 2000. 164: p. 247-256 and Safford, K. M., et al., Neurogenic differentiation of murine and human adipose-derived stromal cells. Biochem Biophys Res Commun, 2002. 294(2): p. 371-9). It has recently been suggested that MSCs from human skin might have the capacity to differentiate into astrocytes after transplantation in mouse brain (Belicchi, M., et al., Human skin-derived stem cells migrate throughout forebrain and differentiate into astrocytes after injection into adult mouse brain. J Neurosci Res, 2004. 77(4): p. 475-86). If it can be shown that complications such as cell fusion have not occurred, it is possible that neuroprotection might be ascribed to astrocytes formed in the CNS. Further, hASCs have immuno-suppressive capabilities similar to bone marrow MSCs (Blancher, et al., abstract 112, ISSCR, 2005). MSC-mediated induction of tolerance could be therapeutic for reduction of graft vs. host disease, rejection, and modulation of inflammation (Aggarwal, S. and M. F. Pittenger, Human mesenchymal stem cells modulate allogeneic immune cell responses. Blood, 2005. 105(4): p. 1815-22). Therefore, hASCs have the potential to protect not only transplanted hESC-derived neuronal lineages, but to protect endogenous motor neurons after patient diagnosis of ALS.

Neuro-supportive cells such as NSCs, astrocytes, and MSCs may be valuable therapeutic tools for improving the outcome of ALS, but studies of endogenous cells that regulate NSC fate in vivo are far from complete. The NSC niche in vivo is highly vascularized and is likely to involve endothelial cells since they release soluble factors that stimulate the self-renewal of NSCs, inhibit their differentiation, and enhance their neuron production (Shen, Q., et al., Endothelial Cells Stimulate Self-Renewal and Expand Neurogenesis of Neural Stem Cells. Science, 2004. 304(5675): p. 1338-1340). Indeed NSCs in vitro and during mouse development produce endothelial cells, directly reinforcing the proposed role of mesenchymal cell role in NSC maintenance and fate specification (Wurmser, A. E., et al., Cell fusion-independent differentiation of neural stem cells to the endothelial lineage. Nature, 2004. 430(6997): p. 350-356).

In one aspect of the present invention, it has been found that human embryonic stem cells (hESC) can be directed towards differentiation into either NSCs, astrocytes or into neurons and oligodendrocytes on feeders of human adipose derived stem cells (hASC), depending on feeder cell density. Therefore, hASC or cells derived from hESC (astrocytes and NSCs) appear to modulate the local niche in vivo to promote differentiation of progenitor cells and potentially to preserve motor neuron function in ALS. Further, bioengineered delivery systems prepared from 3-dimensional growth matrices, such as collagen matrices, improve long-term viability, immunocompatibility and secretion of factors from neuroprotective cells in animal models and patients with neurodegenerative disease (Watts, R. L., et al., Stereotaxic intrastriatal implantation of human retinal pigment epithelial (hRPE) cells attached to gelatin microcarriers: a potential new cell therapy for Parkinson's disease. J Neural Transm Suppl, 2003(65): p. 215-27; Doudet, D. J., et al., PET imaging of implanted human retinal pigment epithelial cells in the MPTP-induced primate model of Parkinson's disease. Exp Neurol, 2004. 189(2): p.361-8 and Bakay, R. A., et al., Implantation of Spheramine in advanced Parkinson's disease (PD). Front Biosci, 2004. 9: p. 592-602). Growth matrices, such as collagen matrices may be evaluated for their ability to improve the functioning of neuroprotective cells. Additionally, growth factors that are supportive of these cells, for example and without limitation, GDNF and CNTF, can be encapsulated for slow release and evaluated for support of motor neurons. The evaluation to select the combination of astrocytes, NSCs and hASCs and growth factor concentrations can first be done in vitro using motor neurons derived from hESCs. In vitro testing will allow direct testing of the necessity and sufficiency of each component. Following in vitro evaluation, transplanted cells within matrices can be optimized in vivo for viability of the transplants in wild type rats. After optimization, these matrices can be evaluated in the ALS rat model with the SOD1 mutation for support of degenerating motor neurons.

The support cell can be a stem cell of mesodermal origin. Mesodermal stem cells generally have the capacity to develop into mesodermal tissues, such as, without limitation, mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures (particularly the oviducts, uterus, and vagina)), pleural and peritoneal tissues, viscera, mesodermal glandular tissues (e.g., adrenal cortex tissues), and stromal tissues (e.g., bone marrow). Inasmuch as the cell can retain potential to develop into mature cells, it also can realize its developmental phenotypic potential by differentiating into an appropriate precursor cell (for example and without limitation, a preadipocyte, a premyocyte, a preosteocyte, etc.). Also, depending on the culture conditions, the cells can also exhibit developmental phenotypes such as embryonic, fetal, hematopoetic, neurogenic, or neuralgiagenic developmental phenotypes.

One non-limiting example of a useful support cell, as mentioned above and as shown in the examples below, is a mesenchymal stem cell derived from adipose tissue. U.S. patent application No. 20020076400, incorporated herein by reference in its entirety for its technical disclosure, describes the isolation of adipose-derived stem cells. U.S. Pat. No. 7,078,230, incorporated herein by reference in its entirety for its technical disclosure, describes derivation of adipose-derived stem cells and describes the potential for differentiation of adipose-derived stem cells into neurons and suggests improvement of spinal cord lesions in rats. From these references and, more generally, from references cited therein, identification of and isolation of mesenchymal stem cells or other suitable support cells from adipose tissue and from other tissue, is well within the abilities of those of skill in the relevant art.

In another embodiment, the support cell can be a differentiated mesodermal cell, such as a fibroblast or an endothelial cell (mesodermal blood vessel cells). In another embodiment as applied to neuronal cell development, a support cell can be an astrocyte. Combinations of support cells also are useful, such as the combination of astrocytes and ASCs for differentiation of neuronal lineages. Specific combinations of progenitor calls and support cells include, without limitation: embryonic stem cell and adult mesenchymal stem cell to produce neuronal stem cells (human); neuronal stem cell and adult mesenchymal stem cell to produce terminally differentiated neuron, astrocyte and oligodendrocyte (human); embryonic stem cell and other mesenchymal support cell (fibroblasts) to produce neuronal lineages; neural stem cells and endothelial cells (mesodermal blood vessel cells) to maintain neural stem cells; embryonic stem cell and other mesenchymal support cell (mouse bone stromal cells) to produce neuronal lineages; and neuronal stem cells and astrocytes (ectodermal support cells) to produce neurons.

As discussed above, protection and localized retention of transplanted cells is also useful in achieving adequate therapeutic value. Transplanted cells have compromised viability due to mechanical forces during injection. In addition, the environmental transition from enriched culture media to a bolus within the injection site with limited delivery of oxygen and nutrients decreases survival of transplanted cells (Del Guerra, S., et al., Entrapment of dispersed pancreatic islet cells in CultiSpher-S macroporous gelatin microcarriers: Preparation, in vitro characterization, and microencapsulation. Biotechnol Bioeng, 2001. 75(6): p. 741-4). Survivability can be improved when growth matrices, such as, without limitation, beads of 3 dimensional collagen matrices are used to provide adequate intercellular spacing. This provides physical protection during mechanical transfer, promotes diffusion of nutrients, and allows cells to maintain a local environment. Collagen is the major component of nerve tissue and has been widely studied as a biological conduit material. Therefore a collagen-based matrix is useful for generating neuronal tissue. For example and without limitation, Cultisphers-S (Percell Biolytica, Å storp, Sweden) can be used for the production of nerve guides for the support of neuronal outgrowth. Chondrocytes transplanted within Cultisphers produced significantly more extracellular matrix after transplantation (Malda, J., et al., Expansion of human nasal chondrocytes on macroporous microcarriers enhances redifferentiation. Biomaterials, 2003. 24(28): p. 5153-61). Similarly, pancreatic islet cells had better access to nutrients, improved glucose-sensitive insulin secretion and immunoprotection after transplantation within Cultisphers than after injection in a bolus (Del Guerra, S., et al., Entrapment of dispersed pancreatic islet cells in CultiSpher-S macroporous gelatin microcarriers: Preparation, in vitro characterization, and microencapsulation. Biotechnol Bioeng, 2001. 75(6): p. 741-4). In studies that compared Cultisphers with other similar products, porous, biodegradable conduits were developed with multiple longitudinally aligned channels comprised of Cultisphers and PCL (Bender, M. D., et al., Multi-channeled biodegradable polymer/CultiSpher composite nerve guides. Biomaterials, 2004. 25(7-8): p. 1269-1278). Cutlisphers enhanced cortical neuron adhesion, viability, and neurite extension as compared to PCL alone. Additionally, the mechanical properties of the PCL/CultiSpher and in vitro degradation rates suggest good biocompatibility (Bender, M. D., et al., Multi-channeled biodegradable polymer/CultiSpher composite nerve guides. Biomaterials, 2004. 25(7-8): p. 1269-1278). Schwann cells (Bender, M. D., et al., Multi-channeled biodegradable polymer/CultiSpher composite nerve guides. Biomaterials, 2004. 25(7-8): p. 1269-1278) and PC12 cells (Waddell, R. L., et al., Using PC12 cells to evaluate poly(caprolactone) and collagenous microcarriers for applications in nerve guide fabrication. Biotechnol Prog, 2003. 19(6): p. 1767-74) attached and proliferated on CultiSphers and PCL/CultiSpher composite materials.

Use of Cultisphers in the treatment of neurological disease has recently been accomplished by the transplantation of dopamine-secreting retinal pigment epithelial cells. Cells attached to cultispher gelatin microcarriers (Spheramine) in rodent and non-human primate models of Parkinson's disease (PD) produces long-term amelioration of motor and behavioral deficits, with histological and PET evidence of cell survival without immunosuppression. Long-term safety in cynomologous monkeys has also been demonstrated (Watts, R. L., et al., Stereotaxic intrastriatal implantation of human retinal pigment epithelial (hRPE) cells attached to gelatin microcarriers: a potential new cell therapy for Parkinson's disease. J Neural Transm Suppl, 2003(65): p. 215-27). Spheramine appears to show promise in treating late stage PD patients. Stereotactic transplantations were well tolerated and patients showed signs of improvement at 1 year (Watts, R. L., et al., Stereotaxic intrastriatal implantation of human retinal pigment epithelial (hRPE) cells attached to gelatin microcarriers: a potential new cell therapy for Parkinson's disease. J Neural Transm Suppl, 2003(65): p. 215-27). Implanted human retinal pigment epithelial cells can survive in the brain without immunosuppression in moderately to severely impaired monkeys with bilateral 1-methyl-4-phenyl-1,2,3,6-tetrahydropyridine hydrochloride (MPTP)-induced parkinsonism (Doudet, D. J., et al., PET imaging of implanted human retinal pigment epithelial cells in the MPTP-induced primate model of Parkinson's disease. Exp Neurol, 2004. 189(2): p. 361-8). Positron emission tomography (PET) with [¹⁸F]fluoro-L-dopa (FDOPA) showed increased accumulation implicating a dopaminergic mechanism of action (Doudet, D. J., et al., PET imaging of implanted human retinal pigment epithelial cells in the MPTP-induced primate model of Parkinson's disease. Exp Neurol, 2004. 189(2): p. 361-8). These studies suggest that Cultisphers are stable in rodent, primate, and human CNS; they support the long-term survival of allogenic stromal cells that secrete diffusible neuroprotective agents, and they provide patients with a delivery system that is effective against neurodegenerative disease (Bakay, R. A., et al., Implantation of Spheramine in advanced Parkinson's disease (PD). Front Biosci, 2004. 9: p. 592-602).

The ability of growth factors to influence neural cell survival is under investigation, and many groups are studying the addition of growth factors to transplantable scaffolds. Slow release PLGA microspheres have been constructed using a double emulsion technique for the delivery of various growth factors including FGF-1, TGF-P and larger proteins (Meese, T. M., et al., Surface studies of coated polymer microspheres and protein release from tissue-engineered scaffolds. J Biomater Sci Polym Ed, 2002. 13(2): p. 141-51; Hu, Y., J. O. Hollinger, and K. G. Marra, Controlled release from coated polymer microparticles embedded in tissue-engineered scaffolds. J Drug Target, 2001. 9(6): p. 431-8; Royce, S. M., M. Askari, and K. G. Marra, Incorporation of polymer microspheres within fibrin scaffolds for the controlled delivery of FGF-1. J Biomater Sci Polym Ed, 2004. 15(10): p. 1327-36 and DeFail, A. J. 2006, et al., Controlled Release of Bioactive TGF-β1 from Microspheres Embedded within Biodegradable Hydrogels. Biomaterials, 27(8): 1579-85). Neurotrophic factors have been incorporated into some of these biomaterials to further improve axonal regrowth. The use of growth factors in nerve regeneration includes the incorporation of glial growth factor (GGF) within a polymer conduit (Bryan, D. J., et al., Influence of glial growth factor and Schwann cells in a bioresorbable guidance channel on peripheral nerve regeneration. Tissue Eng, 2000. 6(2): p. 129-38). GGF is produced by neurons and stimulates proliferation of Schwann cells. The role of NGF in nerve regeneration has been more widely studied. The neurotrophin NGF promotes the differentiation of several classes of neurons, is a survival factor in both neuronal cell culture and in vivo, (Yakovchenko, E., et al., Insulin-like growth factor I receptor expression and function in nerve growth factor-differentiated PC12 cells. J Neurochem, 1996. 67(2): p. 540-8) and has been encapsulated and delivered from polymer microspheres to promote the survival of cholinergic neurons (Pean, J. M., et al., Intraseptal implantation of NGF-releasing microspheres promote the survival of axotomized cholinergic neurons. Biomaterials, 2000. 21(20): p. 2097-101). Tranquillo's group has had promising results with the addition of NGF within guides (Rosner, B. I., et al., Rational design of contact guiding, neurotrophic matrices for peripheral nerve regeneration. Ann Biomed Eng, 2003. 31(11): p. 1383-401). Other neurotrophic factors such as ciliary neurotrophic factor (CNTF) (Tan, S. A., et al., Rescue of motoneurons from axotomy-induced cell death by polymer encapsulated cells genetically engineered to release CNTF. Cell Transplant, 1996. 5(5): p. 577-87) and GDNF (Fine, E. G., et al., GDNF and NGF released by synthetic guidance channels support sciatic nerve regeneration across a long gap. Eur J Neurosci, 2002. 15(4): p. 589-601) delivered from slow release polymer beads, may be more effective than NGF in the preservation of motomeurons after injury. Thus, the development of slow-release growth factors within a stable collagen matrix that supports cells involved in motor neuron protection is a well-grounded strategy that might increase the quality of life for ALS patients.

### EXAMPLE 1

### Growth, Differentiation and Transplantation of hESCs

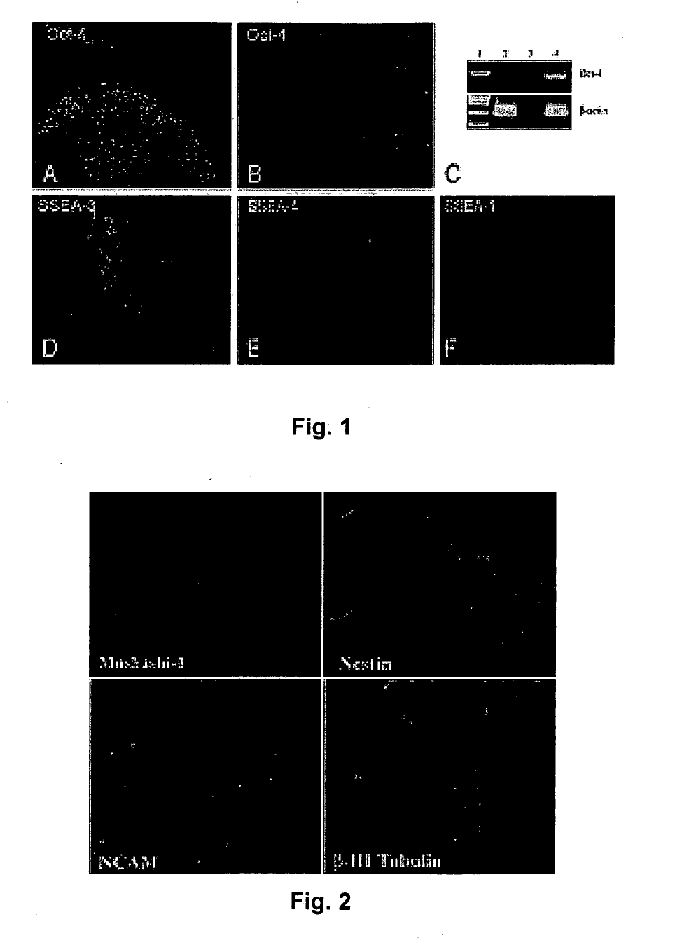

Pluripotent hESCs (HSF-6, University of San Francisco and H7, Wicell). can be maintained on feeder fibroblasts derived from 14.5 day CF-1 mouse embryos (FIG. 1) or FF-2 human foreskin fibroblasts (ATCC) (Mangoubi, R. S., Jeffreys, C. G. Desai, M. N. Jane, E. P. Sammak, P. J, Support Vector Machine and Parametric Wavelet-Based Texture Classification of Stem Cell Images. Submitted IEEE Transactions in Biomedical Engineering, 2005; Sammak, P. J., et al., Pluripotent Embryonic Stem Cells Have Plastic Chromatin and Nuclei that Stabilize Upon Differentiation. Developmental Cell, 2005. submitted; and Constantinescu, D., et al., Lamin A/C expression is an early marker of mouse and human embryonic stem cell differentiation. Stem Cells, 2005. In Press). We have differentiated hESC into neuronal lineages in vitro by several methods. Reducing the density of feeder fibroblasts permits controlled differentiation of HSF-6 hESCs to neuronal lineages including neural stem cells (NSC, nestin and pax-6 positive, not shown) and neurons (Beta III tubulin, NCAM, mushashi 1, FIG. 2).

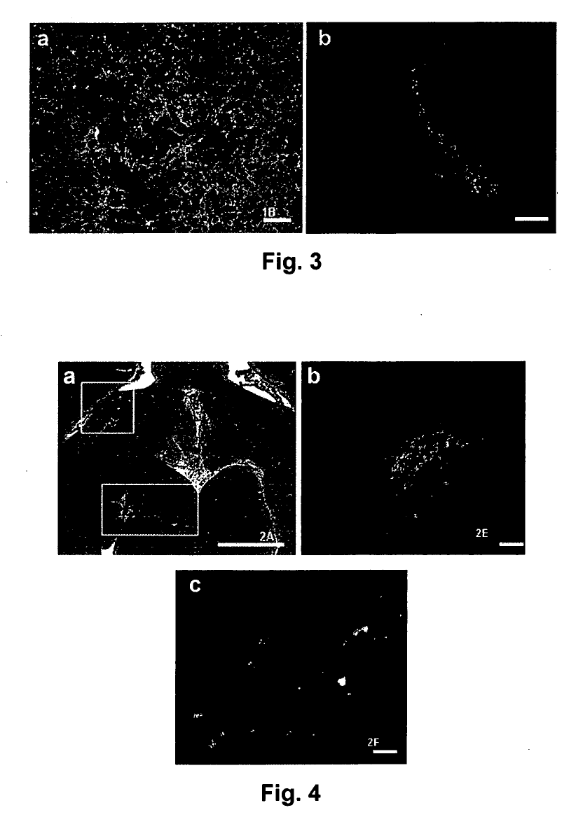

The short-term fate of these cells when transplanted in vivo was evaluated in mouse brains, injected with an 18-gauge needle into stereotactically defined regions of the cortex. At 24 hrs, nuclear fragmentation is very prevalent at the site of injection (FIG. 3) and injected pluripotent hESC can be found distally in the sub-ventricular zone, including Oct-4 positive cells. This experiment highlights three problems. First, acute injury of stem cells after injecting is a significant factor reducing cell viability. Second, stem cells are mobile and travel to favored niches even if distant from the injection site, and third pluripotent cells can survive at least for short times within the sub-ventricular zone. When injecting neuronal cells derived from hESC, precautions must be taken to avoid also injecting contaminating pluripotent cells that could produce teratomas. Nestin-positive NSCs were injected at a location 2 mm caudal to a second injection site where astrocyte-like glioblastoma cells, line U87, were injected (FIG. 4). In vitro, U87 cells secrete several growth factors including FGF2, HGF, TGF-β, IL-6 and others (Todo, T., et al., Expression and growth stimulatory effect of fibroblast growth factor 9 in human brain tumors. Neurosurgery, 1998. 43(2): p. 337-46; Hotfilder, M., et al., Interferon-gamma increases IL-6 production in human glioblastoma cell lines. Anticancer Res, 2000. 20(6B): p. 4445-50; Brockmann, M. A., et al., Glioblastoma and cerebral microvascular endothelial cell migration in response to tumor-associated growth factors. Neurosurgery, 2003. 52(6): p. 1391-9; discussion 1399; Yamada, S. M., et al., Fibroblast growth factor receptor (FGFR) 4 correlated with the malignancy of human astrocytomas. Neurol Res, 2002. 24(3): p. 244-8) and promote hESC differentiation and NSC chemotaxis (not shown). After 5 days, NSCs prelabeled with dyes CMFDA and DiI migrated to the subventricular zone 1 mm away and to the glioblastoma injection site 2 mm distant from the NSC injection site. NSCs were not found at other regions surrounding the injection site. Many of these retained the NSC marker, nestin (not shown). Mobilization of cells is a specific response to the local environment whether due to injury (Lim, S. S., P. J. Sammak, and G. G. Borisy, Progressive and spatially differentiated stability of microtubules in developing neuronal cells. J Cell Biol, 1989. 109(1): p. 253-63; Sammak, P. J. and G. G. Borisy, Direct observation of microtubule dynamics in living cells. Nature, 1988. 332(6166): p. 724-6; Sammak, P. J., et al., How do injured cells communicate with the surviving cell monolayer? J Cell Sci, 1997. 110 (Pt 4): p. 465-75; Unger, G. M., et al., Quantitative assessment of leading edge adhesion: reattachment kinetics modulated by injury-derived intracellular calcium predict wound closure rates in endothelial monolayers. J Cell Physiol, 1998. 174(2): p. 217-31; and Tran, P. O., et al., A wound-induced [Ca²⁺] increase and its transcriptional activation of immediate early genes is important in the regulation of motility. Exp Cell Res, 1999. 246(2): p. 319-26), or local stabilizing niches. Therefore, NSC not only home to endogenous stem cell niches in the subventricular zone, but the local environment can be modified by injection of support cells that provide an artificial niche for stem cells.

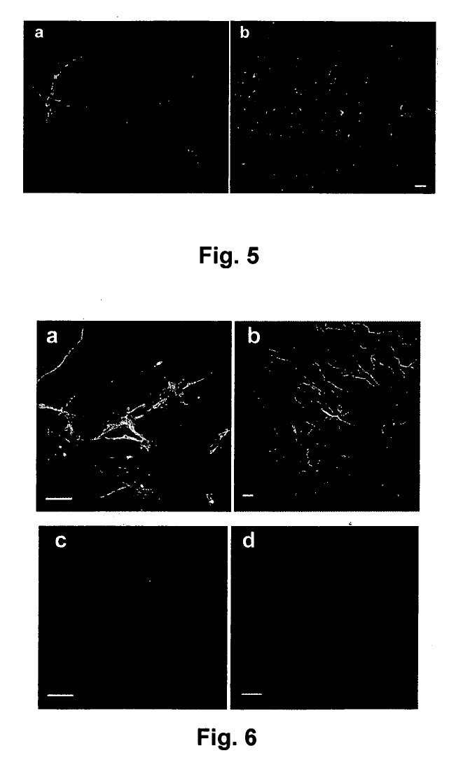

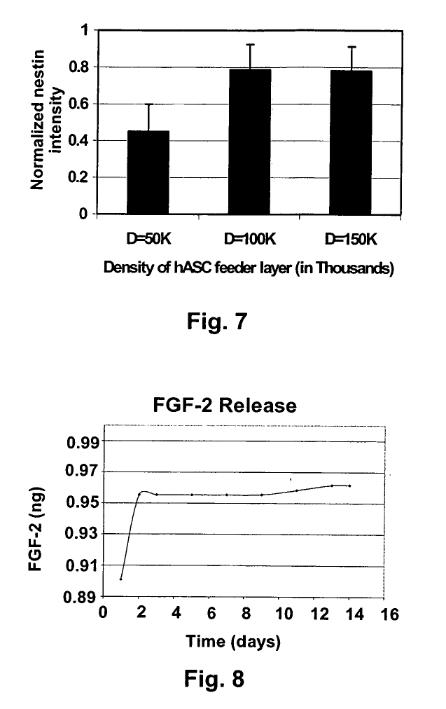

Of course, glioblastomas are not themselves appropriate as support cells, but astroglial lineages might provide a supportive environment. To derive astroglial cells in vitro, we evaluated preadipocytic bone stromal cells from mouse, line MS5 (Barberi, T., et al., Neural subtype specification of fertilization and nuclear transfer embryonic stem cells and application in parkinsonian mice. Nat Biotechnol, 2003. 21(10): p. 1200-7) that produce NSCs that can be further directed into most neuronal lineages in co-culture with an adult stem cell derived from human adipose tissue (hASCs) as the feeder layer. These thy-1 positive MSCs are patient specific and can be used in transplantation studies for soft tissue reconstruction (not shown). To humanize these studies, hESCs, instead of MS5 cells, were co-cultured with hASCs to derive human astroglial cells. It was found that long-term culture of hESCs on high-density hASCs promotes extensive neurite outgrowth, unlike MS5 cells (FIG. 5). At low density, hASCs promote differentiation of neuronal support cells, both astrocytes and oligodendrocytes (FIG. 6). Further, at low hASC density, nestin (FIG. 7) and beta III tubulin are expressed at quantitatively lower density, increasing the percentage of astrocytes and oligodendrocytes in the culture. Further differentiation of these cells at higher purity can be controlled by addition of selective growth factors such as EGF/CNTF to produce astrocytes (Barberi, T., et al., Neural subtype specification of fertilization and nuclear transfer embryonic stem cells and application in parkinsonian mice. Nat Biotechnol, 2003.21(10): p.1200-7) and EGF/PDGF to produce oligodendrocytes (Barberi, T., et al., Neural subtype specification of fertilization and nuclear transfer embryonic stem cells and application in parkinsonian mice. Nat Biotechnol, 2003. 21(10): p.1200-7 and Nistor, G. I., et al., Human embryonic stem cells differentiate into oligodendrocytes in high purity and myelinate after spinal cord transplantation. Glia, 2005. 49(3): p.385-96).

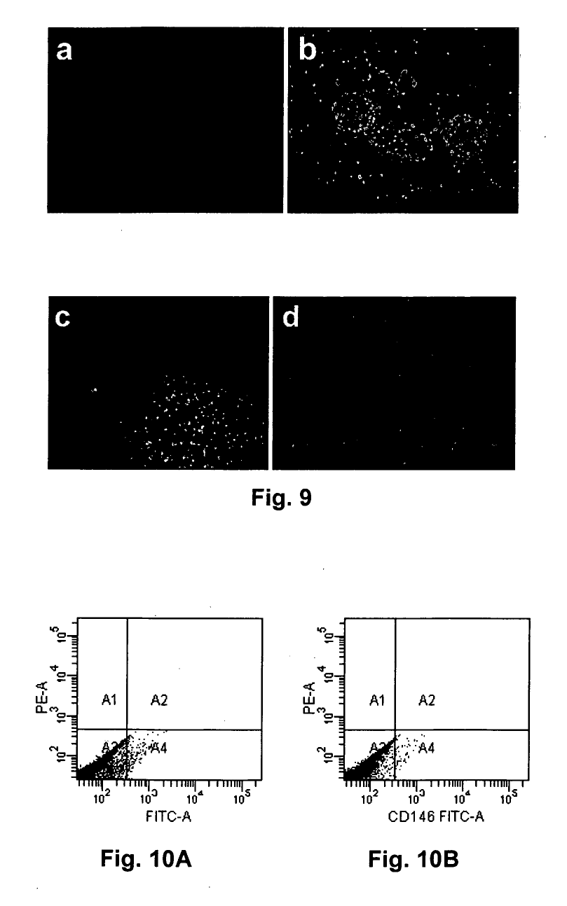

Lineage restriction could be performed in vitro with the addition of growth factor to the media or potentially in vivo by the slow release of growth factor from polymer beads (FIG. 8) that are transplanted in vivo along with NSCs. The controlled release of growth factors from porous, polymer scaffolds has been proposed for use as tissue-engineered scaffolds. We described the use of poly(D,L-lactic-co-glycolic acid) (PLGA) microspheres (see, Meese, T. M., et al., Surface studies of coated polymer microspheres and protein release from tissue-engineered scaffolds. J Biomater Sci Polym Ed, 2002.13(2): p.141-51; Hu, Y., J. O. Hollinger, and K. G. Marra, Controlled release from coated polymer microparticles embedded in tissue-engineered scaffolds. J Drug Target, 2001. 9(6): p. 431-8; Royce, S. M., M. Askari, and K. G. Marra, Incorporation of polymer microspheres within fibrin scaffolds for the controlled delivery of FGF-1. J Biomater Sci Polym Ed, 2004.15(10): p.1327-36 and DeFail, A. J. 2006, et al., Controlled Release of Bioactive TGF-β1 from Microspheres Embedded within Biodegradable Hydrogels. Biomaterials, 27(8):1579-85) for the slow-release of FGF-1, TGF-β1 and larger proteins. Growth factors that direct astrocytes differentiation including EGF, GDNF and CTNF (Barberi, T., et al., Neural subtype specification of fertilization and nuclear transfer embryonic stem cells and application in parkinsonian mice. Nat Biotechnol, 2003.21(10): p.1200-7) or that support motor neuron survival including GDNF (Klein, S. M., et al., GDNF delivery using human neural progenitor cells in a rat model of ALS. Hum Gene Ther, 2005.16(4): p. 509-21) could be used for the first several weeks (FIG. 8). Longer term conditioning of the local microenvironment could be obtained by growth factors secreted by the transplanted NSCs (Llado, J., et al., Neural stem cells protect against glutamate-induced excitotoxicity and promote survival of injured motor neurons through the secretion of neurotrophic factors. Mol Cell Neurosci, 2004. 27(3): p.322-31), astrocytes (Klein, S. M., et al., GDNF delivery using human neural progenitor cells in a rat model of ALS. Hum Gene Ther, 2005.16(4): p.509-21) and the MSCs, hASCs (FIGS. 5-7). Acute protection of transplanted cells can be provided by Cultisphers which provide better control of cell density and nutrient diffusion (Del Guerra, S., et al., Entrapment of dispersed pancreatic islet cells in CultiSpher-S macroporous gelatin microcarriers: Preparation, in vitro characterization, and microencapsulation. Biotechnol Bioeng, 2001. 75(6): p. 741-4). Cultisphers also provide mechanical protection, which is likely to be problematic with friable NSCs (FIG. 3). NSCs, hASCs or astrocytes can be transplanted in vivo in 200 μm Cultisphers-S or in aggregates of Cultisphers prepared as neural guides (FIG. 9) that will support the growth of neurons and oligodendrocytes in vivo (Bender, M. D., et al., Multi-channeled biodegradable polymer/CultiSpher composite nerve guides. Biomaterials, 2004. 25(7-8): p. 1269-1278; Waddell, R. L., et al., Using PC12 cells to evaluate poly(caprolactone) and collagenous microcarriers for applications in nerve guide fabrication. Biotechnol Prog, 2003. 19(6): p. 1767-74 and Marra, K. G., The Use of Polymeric Materials to Construct Biodegradable Nerve Guides. Polymer News, 2003. 28(11): p.343-347). Further, Cultisphers can be derivatized with extracellular matrix proteins to improve immunoprotection (Malda, J., et al., Expansion of human nasal chondrocytes on macroporous microcarriers enhances redifferentiation. Biomaterials, 2003.24(28): p. 5153-61), or adhesion. Laminin improves differentiation of hESC to neuronal lineages and laminin peptide fragments p31 and p20 promote hESC adhesion and neuronal differentiation, respectively (data not shown). However, modification of Cultisphers with laminin or laminin peptides would favor neurons over astrocytes or neural progenitors and thus collagen based Cultisphers are preferred.

Neurons infiltrated into the nerve guides and survived for 12 weeks.

### EXAMPLE 2

### Characterization of hASCs

While ESCs were characterized by the provider (University of California-San Francisco for line HSF6 and WiCell for HI cell line), the ASCs were isolated and characterized specifically for this project from patients. ASCs were identified by differential adhesion used during the cell isolation procedure, the presence of stem cell marker (CD90) and 26, absence of the endothelial cell marker, CD34 and smooth muscle marker CD146). Further, we find that the ESCs can be directed down desired neural, glial or oligodendrocytic lineages by varying the density of ASCs in co-culture

In this Example, two behaviors of ESCs on ASC feeder layers were characterized: the degree of natural ectodermal differentiation by ESCs: neural stem cells versus neural subtypes and the number of hESCs that were driven toward neural differentiation. Human Adipose Stem Cells were isolated through collagenase digestion of whole adipose tissue obtained from patients undergoing elective surgical procedures. Following mechanical dissociation and centrifugation, red blood cells were lysed with erythrocyte lysis buffer and the cells were plated in standard plating media in T75 flasks

Following Isolation, ASC populations were characterized through fluorescence activated cell sorter (FACS) analysis. FACS analysis was performed using antibodies against: CD29 (Integrin Beta 1); CD34; CD49d (Integrin Alpha 4); CD90 (Thy-1); and CD 146 (Mel-CAM). Human embryonic stem cell (hESC) HSF-6, University of San Francisco and H7, Wicell were obtained. These hESC lines are both female ESC cell lines originally passaged on mouse derived feeders in DMEM with high glucose and 20% knock out serum, as recommended by the providers.

After hESCs were expanded in an undifferentiated state, they were transferred to mitomycin-C treated hASC feeder layers of three densities: 50,000, 100,000 or 150,000 cells per 10 cm² well. The cells were then co-cultured for either 19 days or 60 days. No growth factors were added to the culture media. The differentiation of hESCs was analyzed using immunocytochemistry.

FIGS. 10A and 10B show FACS analysis of the ASCs for CD146 (mel-CAM, Muc-18). Vascular smooth muscle cells and endothelial cells are reported to have positive expression by Gronthos et al., 2001 (J. Cell Physiol. 2001 189(1); 54-63). ASCs appeared to be negative of marker CD146, otherwise known as mel-CAM. This is a cell surface glycoprotein that is present on vascular smooth muscle cells and endothelial cells. The FITC control used showed false positive which was also seen in the CD146 single color compensation.

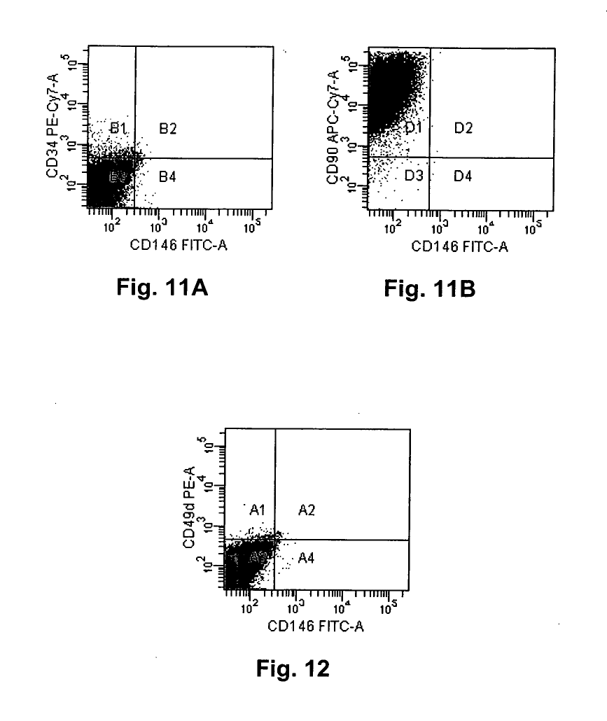

FIGS. 11A and 11B show FACS analysis of the ASCs for CD34 and CD90 (Thy-1), respectively; the latter is indicative of a less-differentiated mesenchymal cell FIG. 12 shows FACS analysis of the ASCs for CD49d. 1-2 % of ASCs were positive for CD49d. This integrin was shown by both Katz et al (Clin Plastic Surg. 1999 26(4); 587-603) and Gronthos (J. Cell Physiol. 2001 189(1); 54-63) to be expressed by the ASCs, though they had conflicting percentages. This marker has been shown to be negative on mesenchymal stem cells.

TABLE A
summary of FACS analysis of ASCs,

| Antigen | % positive |
|---|---|
| Less differentiated cell phenotypes | 96-99.7% |
| CD34(−)CD90(+) | |
| Less differentiated fibroblasts or MSCs | 36.2% |
| CD29(+)CD90(+) | |
| Highly proliferative endothelial cells | 1-2% |
| CD34(+)CD90(+) | |
| Receptor for fibronectin and VCAM-1 | 1.6% |
| CD49d(+)CD29(+) | |

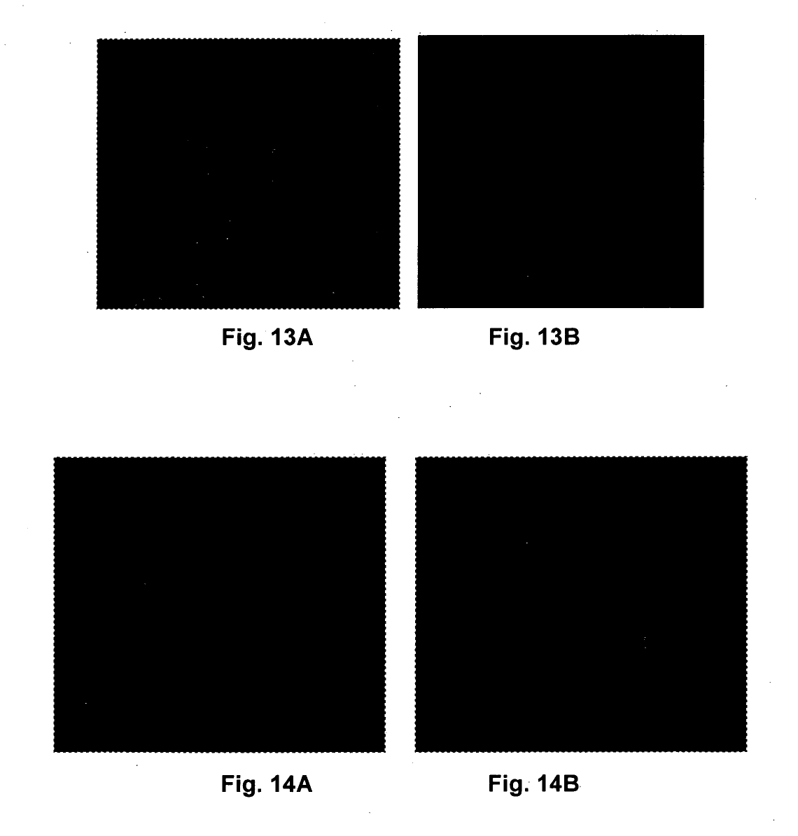

To evaluate completion of neuroectoderm differentiation of hESCs on human ASCs, residual ESCs were revealed by anti-Oct4 (Pluripotency marker) antibody immunostaining following the 19 day co-culture period. FIGS. 13A and 13B are photomicrographs showing the result of this experiment. There was no detectable expression of Oct4 from ESCs cultured with hASCs, therefore all hESCs differentiated past the teratoma forming stage. However, hESCs cultured with human fibroblasts as a control retained some undifferentiated cell populations as determined by the presence of Oct4. Feeder density was 50,000 cells per 10 cm² well in a 6-well plate.

Some hESCs retained early neural stem cell qualities. FIGS. 14A and 14B provide photomicrographs of the hESCs immunostained with anti-Pax6 antibody (early neurectoderm marker). Pax6 expression can be seen in both the control fibroblast feeder layers as well as the ASC feeder layers. The detection of Pax6 expression increased with ASC feeder density. No expression could be detected at 50,000 cells per 10 cm² well. Highest expression was seen in this image at 150,000 cells per 10 cm² well.

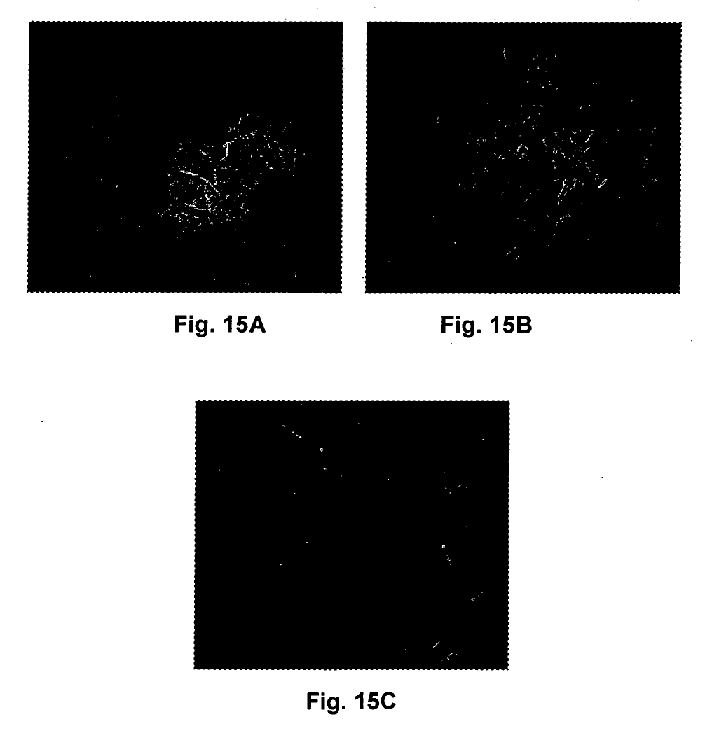

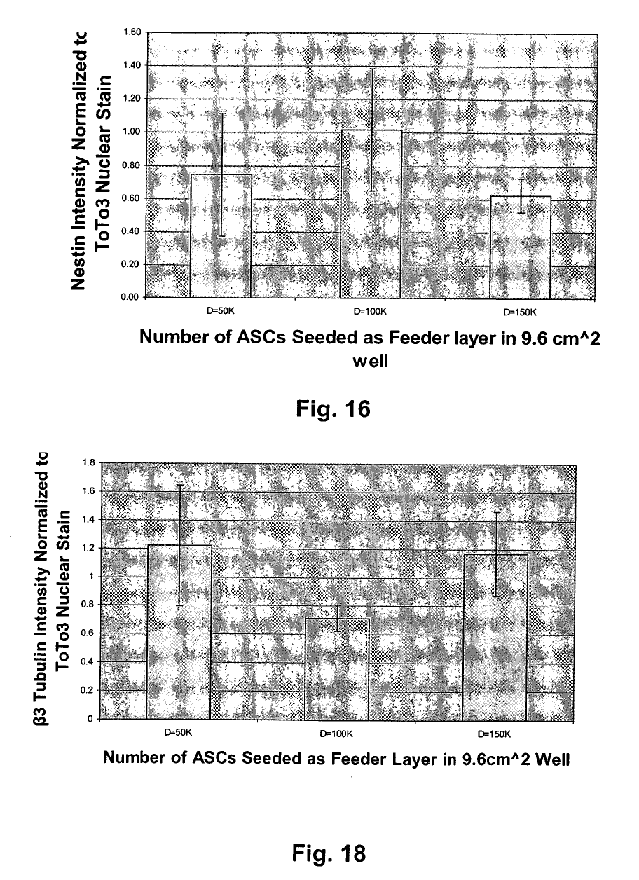

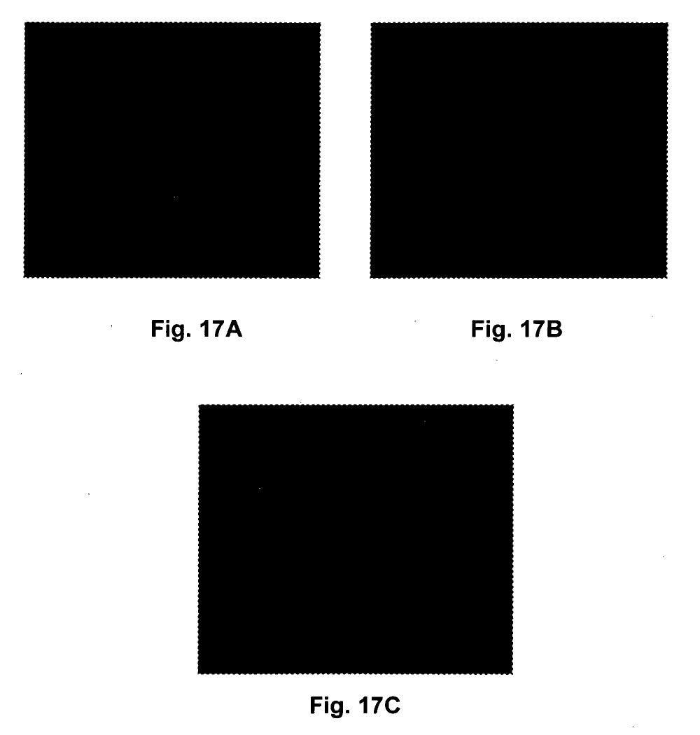

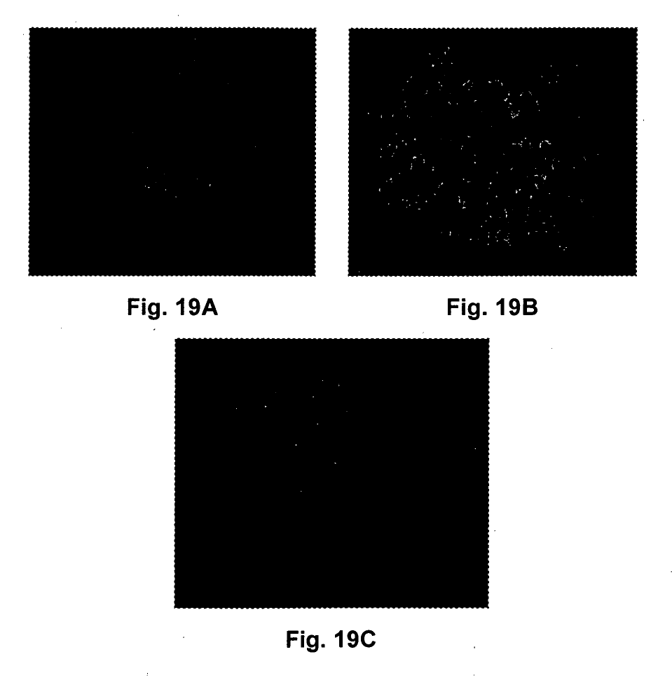

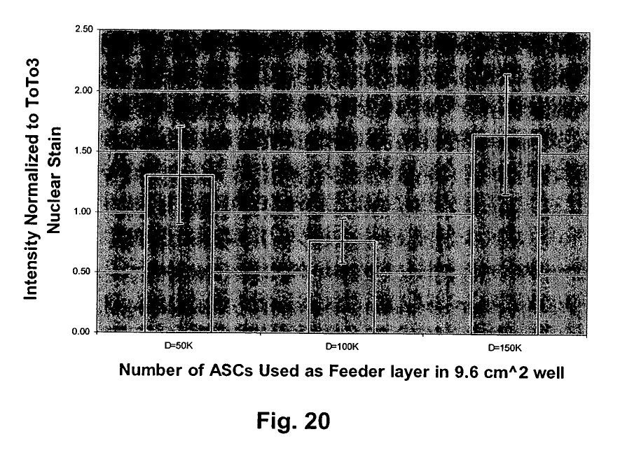

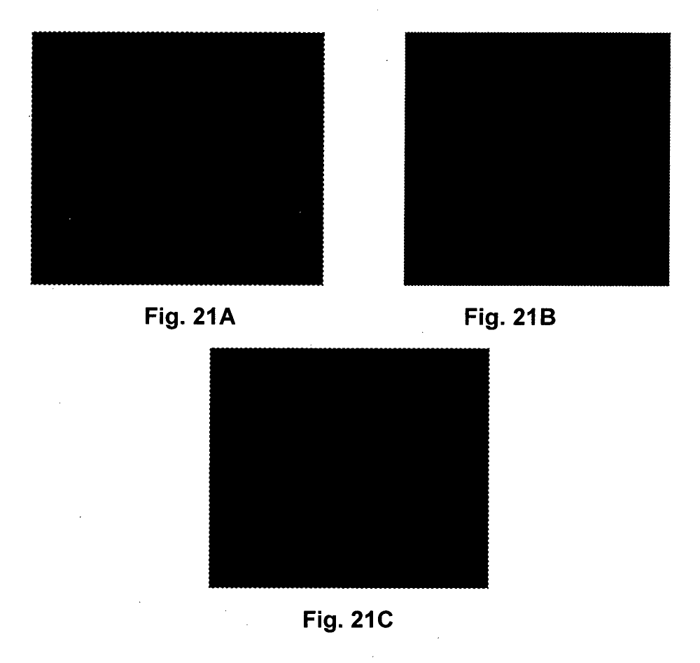

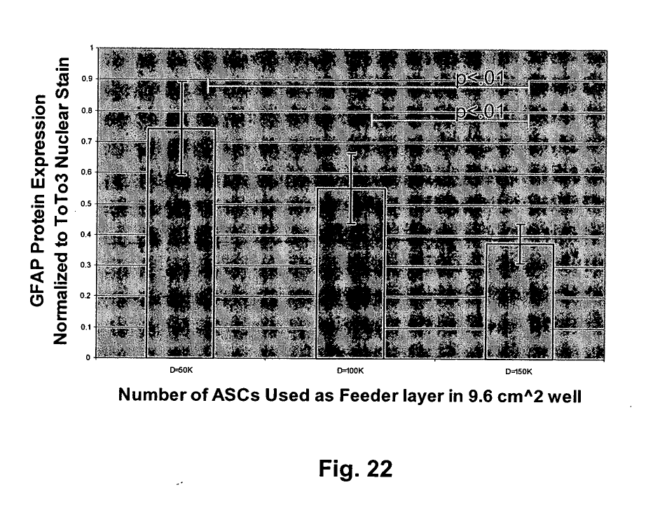

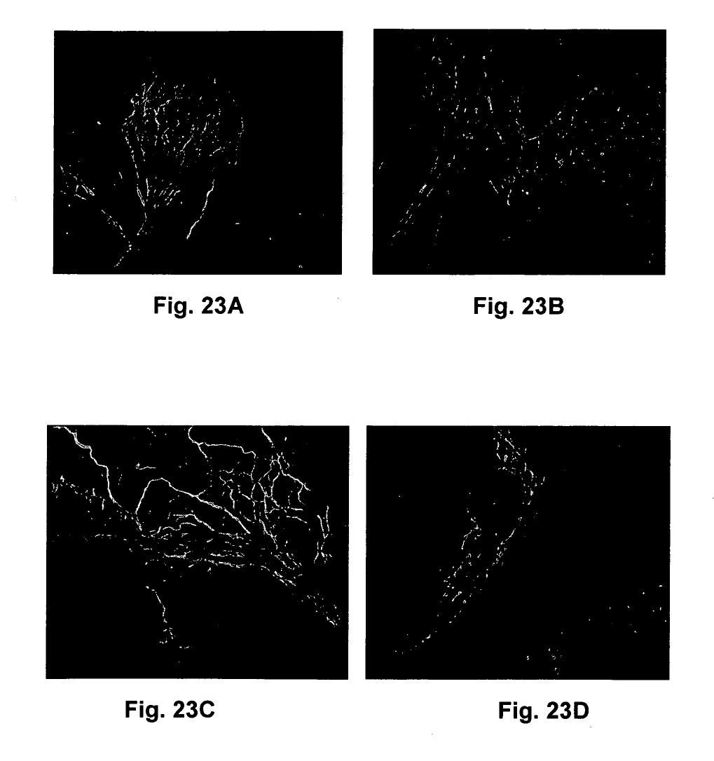

To derive astroglial cells in vitro, we evaluated preadipocytic bone stromal cells from mouse, line MS5, that produce NSCs that can be further directed into most neuronal lineages in co-culture with an adult stem cell derived from human adipose tissue (hASCs) as the feeder layer. These thy-1 positive MSCs are patient specific and can be used in transplantation studies for soft tissue reconstruction (not shown). To humanize these studies, hESCs, instead of MS5 cells, were co-cultured with hASCs to derive human astroglial cells. Co-culture of hASCs and human embryonic stem cells (hESCs) has not been previously evaluated for its influence on fate selection of hESCs, to the best of our knowledge. At low density hASCs promote differentiation of neuronal support cells, both astrocytes and oligodendrocytes (FIG. 6). Further, at low hASC density, nestin (FIGS. 15A-C and 16) and beta III tubulin (FIGS. 17A-C) are expressed at quantitatively lower density, increasing the percentage of astrocytes (FIGS. 21 A-C, 22) and oligodendrocytes (FIG. 19A-C and 20) in the culture. Further differentiation of these cells at higher purity can be controlled by addition of selective growth factors such as EGF/CNTF to produce astrocytes (Barberi, T., et al., Neural subtype specification of fertilization and nuclear transfer embryonic stem cells and application in parkinsonian mice. Nat Biotechnol, 2003. 21(10): p.1200-7) and EGF/PDGF to produce oligodendrocytes (Barberi, T., et al., Neural subtype specification of fertilization and nuclear transfer embryonic stem cells and application in parkinsonian mice. Nat Biotechnol, 2003.21(10): p.1200-7 and Nistor, G. I., et al., Human embryonic stem cells differentiate into oligodendrocytes in high purity and myelinate after spinal cord transplantation. Glia, 2005.49(3): p.385-96). Further, long-term culture of hESCs on high-density hASCs promotes extensive neurite outgrowth, unlike MS5 cells (FIG. 23A and 23B), suggesting that ASCs are effective for promoting neural elongation and synapseformation.

### EXAMPLE 3

We have differentiated human embryonic stem cells (hESCs) into neural stem cells (NSCs), neurons, astrocytes and oligodendrocytes in a hESC-hASC (Patient-specific human adipose tissue-derived stem cell) co-culture system. NSCs are neuroprotective and hASC are immunosuppressive. This study describes a 3-D collagen matrix containing an optimized combination of NSC, hASC, astrocytes, and growth factors that will protect motor neurons in an animal model of ALS from neurodegeneration without generating an extensive immune response. This study involves the following tasks.

Develop a humanized neural stem cell culture based on co-culture of hESC on hASC 086]Determine hASC concentration and culture media composition for optimal production of NSC and astrocytes in a humanized system that will be minimally immunogenic in humans.

Differentiate NSC into motor neurons to be used for in vitro testing of matrices and characterize by marker expression and electrophysiological function and by absence of the non-human antigen Neu5Gc.

Develop collagen matrices for hASCs, hESC-derived NSCs, and astrocytes and develop polymer encapsulated neurotrophic factors.

Co-culture hASC and hESC in collagen matrices to produce NSC-hASC or astrocytes-hASC matrices.

Encapsulate the growth factors GDNF, CNTF and FGF-2 in slow release PLGA microspheres

Evaluate NSC-hASC and astrocyte-hASC matrices as well as growth factor microspheres for transplant viability and motor neuron support.

Test in vitro effects of NSC-hASC and astrocyte-hASC matrices as well as growth factor microspheres on motor neuron growth.

Optimize survival, differentiation, and integration of transplanted NSC-hASC astrocytes-hASC matrices and growth factor microspheres in WT rat spinal cord.

Test motor neuron support by transplanted NSC-hASC astrocyte-hASC matrices and growth factor microspheres in SOD 1 rat model of spinal cord degeneration.

Develop a humanized neural stem cell culture based on co-culture of hESC on hASC

Determine HASC concentration and culture media composition for optimal production of NSC and astrocytes in a humanized system that will be minimally immunogenic in humans.

Differentiate NSC into motor neurons to be used for in vitro testing of matrices and characterize by marker expression and electrophysiological function and by absence of the non-human antigen Neu5Gc.

The rationale for these experiments is to derive stem cell products that can be tested for neuroprotection, including NSC and astrocytes from hESC. Patient-specific hASC can be collected for derivation of hESC and for transplantation. Motor neurons can be derived from hESC not for transplantation, but for in vitro testing of neural support by hASCs, astrocytes, and NSCs. The experiments described herein show that humanized culture conditions (media and feeder cells) are feasible for use in differentiation of hESCs to NSCs and neuronal lineages using patient specific hASCs as feeders. As described above, these protocols are adaptations of established protocols with mouse preadipocyte cells (Barberi, T., et al., Neural subtype specification of fertilization and nuclear transfer embryonic stem cells and application in parkinsonian mice. Nat Biotechnol, 2003. 21(10): p. 1200-7) for human, (Perrier, A. L., et al., Derivation of midbrain dopamine neurons from human embryonic stem cells. Proc Natl Acad Sci U S A, 2004. 101(34): p. 12543-8) and non-human primate (Takagi, Y., et al., Dopaminergic neurons generated from monkey embryonic stem cells function in a Parkinson primate model. J. Clin. Invest., 2005. 115(1): p. 102-109) ESCs. The preadipocyte protocols are robust, and produce stable, multipotent neuronal stem cells from multiple species and lineages of ESCs (Barberi, T., et al., Neural subtype specification of fertilization and nuclear transfer embryonic stem cells and application in parkinsonian mice. Nat Biotechnol, 2003. 21(10): p. 1200-7; Perrier, A. L., et al., Derivation of midbrain dopamine neurons from human embryonic stem cells. Proc Natl Acad Sci U S A, 2004. 101(34): p. 12543-8 and Takagi, Y., et al., Dopaminergic neurons generated from monkey embryonic stem cells function in a Parkinson primate model. J. Clin. Invest., 2005. 115(1): p. 102-109). NSC created on these substrates can be removed from the preadipocyte feeder cells, and cultured in defined media to produce high yield cultures of motor neurons (Li, X.-J., et al., Specification of motoneurons from human embryonic stem cells. Nat Biotechnol. February 2005;23(2):215-21. Epub Jan. 30, 2005). Nevertheless, culture of hESC on mouse feeder cells and/or with animal derived “serum replacers” can transfer non-human sialic acid, Neu5Gc which causes complement-induced killing in human serum (Martin, M. J., et al., Human embryonic stem cells express an immunogenic nonhuman sialic acid. Nat Med, 2005. 11(2): p. 228-32).

Culture of hESC: HES cells, line HSF-6 from UCSF and H7 from Wisconsin were maintained in DMEM high glucose with 20% Knock-Out Serum replacer and passaged as recommended by the provider. HESC were grown on mitomycin-treated human fibroblasts. Colonies were passaged mechanically in clusters of 50-100 cells on feeder layers. Media was changed every other day and colonies were passaged weekly at a 1:2 to 1:3 split. Because these cells were derived on mouse feeder cells, baseline concentrations of non-human antigen NeuSGc will be established as described.

General Methods

Humanized culture protocol: Components for hESC maintenance include: I) Serum Substitute Supplement (SSS; Irvine Scientific) (Weathersbee, P. S., T. B. Pool, and T. Ord, Synthetic serum substitute (SSS): a globulin-enriched protein supplement for human embryo culture. J Assist Reprod Genet, 1995. 12(6): p.354-60 and Lavoir, M. C., J. Conaghan, and R. A. Pedersen, Culture of human embryos for studies on the derivation of human pluripotent cells: a preliminary investigation. Reprod Fertil Dev, 1998. 10(7-8): p. 557-61). SSS affects human embryo maturation similarly to human serum albumin (Graham, M. C., et al., A prospective comparison of Synthetic Serum Substitute and human serum albumin in culture for in vitro fertilization-embryo transfer. Fertil Steril, 1995. 64(5): p. 1036-8) 2) Nutridoma-CS (Roche Applied Science) worked as well as FBS in synthetic 3D culture of chondrocytes (Yates, K. E., F. Allemann, and J. Glowacki, Phenotypic analysis of bovine chondrocytes cultured in 3D collagen sponges: effect of serum substitutes. Cell Tissue Bank, 2005. 6(1): p. 45-54). 3) X-vivo 15 and 20 (Cambrex) are defined medium that contains proteins of human, not animal origin, and have and established FDA file. X-Vivo 15 has been used successfully for feeder free and condition media free culture of hESC with added FGF-2 and noggin (Geron Corporation, ISSCR abstract 2005) and will be our first choice for evaluation. NSCs cultured in humanized and control (MS5 cells, KO serum replacer) protocols will be an evaluated for animal antigens with anti Neu5Gc antibody (Martin, M. J., et al., Human embryonic stem cells express an immunogenic nonhuman sialic acid. Nat Med, 2005. 11(2): p. 228-32) by flow cytometry. Blocking experiments will be performed using chimpanzee serum rich in Neu5Gc (Martin, M. J., et al., Human embryonic stem cells express an immunogenic nonhuman sialic acid. Nat Med, 2005.11(2): p. 228-32). We will defer evaluation of alternative enzymes for HASC isolation.

Isolation of hASC and Characterization: Discard adipose tissue will be collected from patients undergoing elective surgical procedures in the Department of Plastic Surgery, University of Pittsburgh Medical Center. Cells will be isolated from the harvested tissue through collagenase digestion (3.5% Bovine Serum Albumin and 3mg collagenase type II per g of tissue in HBSS) followed by filtration in a double layer of sterile gauze to remove debris. Centrifugation will be performed at 1000 rpm for ten minutes to remove oils, and the pellet will be resuspended in erythrocyte lysis buffer. Centrifugation will be repeated and the remaining stromal vascular fraction will be resuspended in plating medium (DMEM/F 12, 1% Pen/Strep, 0.1 μM Dexamethasone, and 10% FBS). Each derived cell line will be characterized using fluorescence activated cell sorter (FACS) using markers against the following antibodies: CD34, Thy 1, CD29 (integrin β-1), CD49d (integrin α-4), CD90 (expressed on hematopoietic progenitor cells), CD146 (an endothelial cell marker) and NG2 (a pericyte marker).

Differentiation of hESC: Differentiation on preadipocytes has advantages over embryoid body protocols in purity, expandability and stability. Culture on mouse preadipocytes produces a stable, NSC (Pax6+/engrail+/Sox1-) that is stable for at least one year, can be expanded more than 20 fold and provides stock for differentiated neurons for transplantation without teratoma formation. Motor neuron differentiation from NSCs involves sequential treatment with retinoic acid (RA) and sonic hedgehog (SHH) in N2 media, followed by culture in brain derived neurotrophic factor (BDNF) with some variation among protocols (Barberi, T., et al., Neural subtype specification of fertilization and nuclear transfer embryonic stem cells and application in parkinsonian mice. Nat Biotechnol, 2003. 21(10): p. 1200-7; Perrier, A. L., et al., Derivation of midbrain dopamine neurons from human embryonic stem cells. Proc Natl Acad Sci U S A, 2004.101(34): p. 12543-8 and Martin, M. J., et al., Human embryonic stem cells express an immunogenic nonhuman sialic acid. Nat Med, 2005. 11(2): p. 228-32). Alternately, we will evaluate a second protocol (Li, X.-J., et al., Specification of motoneurons from human embryonic stem cells. Nat Biotechnol. February 2005;23(2):215-21. Epub Jan. 30, 2005), where NSC are first treated with retinoic acid (0.001-1 μM) and FGF2 (1-100 ng/ml) followed by a neuronal differentiation medium containing N2 supplement, cAMP (1 μM), retinoic acid (0.1 μM) and SHH (500 ng/ml, R&D) for one week, followed by BDNF, GDNF and IGF1 (10 ng/ml) and SHH reduced to 50 ng/ml.

Immunocytochemistry and Statistical Analysis: Cells are fixed by addition of either 1) 100% methanol −20° C. for 15 min followed by a 15 min wash in PBS+1% Triton X-100 (PBS-Tx, Sigma, St. Louis Mo.) for 15 min, or 2) 2% paraformaldehyde in PBS for 40 min. After blocking with 3% non-fat dry milk, primary antibodies are diluted in PBS+Tx and incubated for 40 min at 37° C., followed by fluorescently labeled secondary antibody for 40 min. 5 μM TOTO-3 (Molecular Probes, Eugene Oreg.) is added and mounted in Vectashield (Vector Labs, Burlinghame, N.H.). Antibodies used for neuronal characterization are detailed in (Li, X.-J., et al., Specification of motoneurons from human embryonic stem cells. Nat Biotechnol. February 2005; 23(2):215-21. Epub Jan. 30, 2005). Measurement of differentiation markers will be evaluated on the single cell level in 3 colonies in each of 3 independent experiments by confocal microscopy and analysis performed with Metamorph (Universal imaging). Standard errors and t-tests (see FIG. 2) will be used. Primary antibodies and the expected tissue distribution are listed below. Antibodies that have not been used by our laboratory in characterizing stem cell differentiation are marked with “ND” for the dilution.

| Antigen | Location | Antibody | Source | Number | Dilution |
|---|---|---|---|---|---|
| 3CB2 | Radial Glia after injury | Mouse monoclonal | U Iowa Dev Bank | 3CB2 | ND |
| A2B5 | Membrane Oligo Precursor | Mouse monoclonal | Chemicon | Mab312R | 1:200 |
| B-1 Integrin | Fibroblasts, Platelets, T cells, | Human Monoclonal | BD Pharmingen | 559883 | 1:200 |
| (CD29) | Mesenchymal Stem | | | | |
| β III tubulin | Early neuronal | Mouse monoclonal | Covance | PRB-435P | 1:100 |
| CD11b | Macrophage and microglia | Mouse monoclonal | Chemicon | CBL1512 | ND |
| CD 34 | Hematopoietic Progenitors, Endothelial | Mouse Monoclonal | Santa Cruz | Sc-7324 | 1:200 |
| | Cells, hASC | | | | |
| DoubleCortin | Migrating early neurons | Guinea pig | Abcam | Ab10293 | 1:1500 |
| (DCX) | | | | | |
| Engrail-1 | Neurectoderm | Mouse Monoclonal | U Iowa Dev Bank | 4G11 | 1:100 |
| ChAT | Choline Aceytyltransferase | Rabbit Polyclonal | Chemicon | AB143 | ND |
| GFAP | Glial Fibrilary Acidic P | Rabbit polyclonal | Calbiochem | AB 5804 | 1:200 |
| GDNF | Glial Growth Factor | Sheep polyclonal | Chemicon | AB5252 | ND |
| GATA6 | Viceral Endoderm | | Santa Cruz | | ND |
| HB9 | Motor neuron | Rabbit polyclonal | Chemicon | AB5963 | 1:200 |
| hnuclei | human nuclear antigen | Mouse monoclonal | Chemicon | MAB1281 | 1:100 |
| Integrin α4 | hASCs, Leukocytes and Erythrocyte | Mouse Monoclonal | eBioscience | 12-0499 | 1:200 |
| | Precursors | | | | |
| mushashi-1 | Neuronal progenitor | Rabbit polyclonal | Chemicon | AB5977 | 1:200 |
| NCAM | Neural progenitor | Rabbit polyclonal | Chemicon | CB5032 | 1:50 |
| Nanog | Pluripotent nuclei | Goat polyclonal | R&D systems | AF1997 | 1:100 |
| nestin | Neural progenitor | Mouse Monoclonal | Covance | MMS570P | 1:100 |
| nestin | Neural progenitor | Rabbit polyclonal | Ab Cam | AB 7659 | 1:100 |
| NG-2 | Chondroitin Sulfate PG on O-2A | Rabbit | Abcam | Ab5320 | 1:100 |
| | progenitors | | | | |
| Neu5Gc | Animal surface sialic acid | Chicken polyclonal | | | 1.5 μg/100 μl |
| NeuN | Neuron specific | Mouse monoclonal | Chemicon | Mab377 | 1:100 |
| O-4 | Oligodendrocyte membrane | Mouse monoclonal | Chemicon | Mab345 | 1:100 |
| Oct-4 | Pluripotent nuclei | Goat polyclonal | R&D systems | MAB1759 | 1:100 |
| Oct-4 | Pluripotent nuclei | Mouse monoclonal | Santa Cruz | SC-5279 | 1:100 |
| Olig-1 | Nuclear oligo-glial precursor | Rabbit polyclonal | Chemicon | AB5991 | 1:200 |
| Pax-6 | Neurectoderm, NSC | Rabbit polyclonal | Covance | PRB 278P | 1:100 |
| PE-CAM | Endothelial progenitor | Mouse monoclonal | Chemicon | MAB2148 | 1:100 |
| PSD95 | Post synaptic Vesicles | Goat Polyclonal | Abcam | ab12093 | 1:100 |
| RC-2 | Radial Glia | Mouse Monoclonal | U Iowa Dev Bank | RC-2 | ND |
| Sox-1 | Late Neural Stem Cell | Rabbit polyclonal | Chemicon | AB4768 | 1:200 |
| SSEA-3 | Pluripotent stem cells | Mouse monoclonal | U Iowa Dev Bank | MC-631 | 1:25 |
| Synaptophysin | Pre-synaptic junction vesicles | Mouse monoclonal | Chemicon | MAB329 | 1:200 |
| Thy-1 | Hematopoetic & hASCs | Mouse Monoclonal | BD Pharmingen | 555594 | 1:200 |
| VAChT | Vesicular Acetylcholine Transporter | Guinea Pig | Chemicon | AB1588 | ND |
| | | polyclonal | | | |

PCR amplification: RNA will be isolated from 5 million human embryonic stem cells pelleted at 800×g for 15 min. 1 ml of Trizol reagent (Invitrogen) will be added to the pellet precipitated with chloroform, pelleted at 12,000 g and dried with ethanol. To produce cDNA for PCR, 1 μg of total RNA will be incubated with dNTP, 1 unit/μl ribonuclease inhibitor, 15 unit/μg AMV RT, 0.5 μg random hexamers, and nuclease free water to a 20 μl final volume incubated at 42° C. for 10 m. The detailed primer sequences are detailed in Li et al (Li, X.-J., et al., Specification of motoneurons from human embryonic stem cells. Nat Biotechnol. February 2005; 23(2):215-21. Epub Jan. 30, 2005). PCR reaction mix (200 μM dNTP, 1.0 μM of each primer, 1.5 mM of MgCl₂, 0.1 volume of 10× reaction buffer, and 1 unit of Taq polymerase) is added to each sample. The cycles are 94° C. for 2 min, 55° C. for 2 min and 72° C. for 2 min. After 30 cycles, the PCR products will be separated by electrophoresis on a 2% agarose gel.

Elecrophysiological recording of neurons: The perforated-patch clamp technique will be employed to study sodium, calcium and potassium currents as previously described (Yazejian, B., et al., Direct measurements of presynaptic calcium and calcium-activated potassium currents regulating neurotransmitter release at cultured Xenopus nerve-muscle synapses. J Neurosci, 1997. 17(9): p. 2990-3001). External bath solution for channel isolation will include (in mM): for calcium current isolation (100 NaCl, 2 KCl, 3 CaCl2, 1 MgCl₂, 30 mM glucose, and 25 HEPES, plus 1 μM TTX—to block sodium current, and 25 mM TEA-Cl—to block potassium currents); for sodium current isolation (100 NaCl, 50 TEA-Cl, 10 HEPES, 5 KCl, 2CaCl₂, and 5 MgCl₂, plus 100 μM CdCl₂—to block calcium currents); and for potassium current isolation (100 NaCl, 2 KCl, 3 CaCl₂, 1 MgCl₂, 30 mM glucose, and 25 HEPES, plus 1 μM TTX—to block sodium currents, and 100 μM CdCl₂—to block calcium currents). All will be at pH 7.3, and adjusted to 310 mOsm with glucose. Borosillicate glass pipettes will be pulled on a Flaming/Brown Micropipette Puller (Sutter Instruments, Model P-97), coated with Sylgard (Dow Corning), and fire polished to a diameter with a measured electrical resistance of 1-3 MOhms. Pipette solution will include (in mM) 75 K₂SO₄, 55 KCl, 10 HEPES, 4 EGTA, and 0.5 CaCl₂, with a pH 7.3. Data will be collected using an Axopatch 200A or 200B amplifier and the pClamp 8.0 software package (Axon Instruments) running on a PC. In all cases currents will be evoked by 100 msec voltage steps from a holding potential of -60 mV to varying voltages between −50 and +60 mV. Data will be analyzed by plotting current-voltage relationships, measuring peak current amplitude, and determining current activation and deactivation kinetics. Furthermore, to confirm the identity of currents, we will use sensitivity to selective reagents that block each in isolation: w-CgTX GVIA (for N-type calcium currents), w-Aga-IVA (for P/Q type calcium currents), nitrendipine (for L-type calcium currents), TTX (for sodium currents), 3,4-diaminopyridine or TEA (for voltage-gated potassium currents), and IBTX for (calcium-activated potassium channels).

Encapsulation of Growth Factors: Growth factors, such as FGF, will be encapsulated into PLGA microspheres using a double emulsion technique, previously described by our lab (Meese, T. M., et al., Surface studies of coated polymer microspheres and protein release from tissue-engineered scaffolds. J Biomater Sci Polym Ed, 2002. 13(2): p.141-51; Hu, Y., J. O. Hollinger, and K. G. Marra, Controlled release from coated polymer microparticles embedded in tissue-engineered scaffolds. J Drug Target, 2001. 9(6): p. 431-8; Royce, S. M., M. Askari, and K. G. Marra, Incorporation of polymer microspheres within fibrin scaffolds for the controlled delivery of FGF-1. J Biomater Sci Polym Ed, 2004. 15(10): p. 1327-36 and DeFail, A. J. 2006, et al., Controlled Release of Bioactive TGF-β1 from Microspheres Embedded within Biodegradable Hydrogels. Biomaterials, 27(8):1579-85) for FGF-1, TGF-β1 and others laboratories for encapsulation of FGF-2 and IGF-1 (Yuksel, E., et al., Increased free fat-graft survival with the long-term, local delivery of insulin, insulin-like growth factor-I, and basic fibroblast growth factor by PLGA/PEG microspheres. Plast Reconstr Surg, 2000. 105(5): p. 1712-20), and for BDNF (Mittal, S., A. Cohen, and D. Maysinger, In vitro effects of brain derived neurotrophic factor released from microspheres. Neuroreport, 1994. 5(18): p. 2577-82). Briefly, 250 mg PLGA will be dissolved in 2 mL of MC. Next, 100 μL of a 10 μg/mL solution of growth factor will be added to 1 mL of a 1% PVA solution, and the solutions will be mixed. The entire mixture will be emulsified on a vortexer for 1 min. The solution will be re-emulsified in 50 mL of 0.1% aqueous PVA solution, resulting in a double emulsion. Next, 100 mL a 2% aqueous isopropanol solution will be added to the second emulsion and stirred for 2 h. The microspheres will be collected by centrifugation, lyophilized to dryness, and stored at −20° C.

Animal Studies: Animal studies will be conducted in accordance with the Principles of National Institute of Health Guide for the Care and Use of Laboratory Animal Care. Rats will be used sparingly (4 per condition paired with control, approximately 24 animals per stage) after in vitro evaluation of Cultispher preparations of stem cells and in vitro evaluation on support of motor neurons prepared by derivation from hESC. After in vitro characterization of transplants, wild type animals (Sprague Dawley) will initially be used to optimize the survival of the grafts (8 conditions×4 animals/condition×3 replicates=96 animals at first and subsequently SOD1 mutants can be grafted prior to appearance of symptoms with the optimized transplants (2 conditions×4 animals/condition×3 replicates=24 animals). Tac:N:(SD)-TgN(SOD1G93A)L26H rats and WT rats can be purchased from Takonic (Germantown, N.Y.). We will evaluate the efficacy of optimized graft preparations on graft viability and motomeuron survival at day 30, since preliminary experiments have shown that stem cell co-cultures produce targeted lineages before that time. SOD1G93A rats can be anesthetized with isoflurane and the lumbar vertebrae can be exposed and clamped in a spinal stereotaxic frame (Kopf Instruments, Tujunga, Calif.). Injection holes can be drilled in laminae within L1 and L4 spinal cord segments and 1-10 μl cell suspension or vehicle can be injected 1.5 mm ventral from dorsal dura surface using a Hamilton syringe outfitted with a 100-200 μm tipped micropipette (Nikkhah, G., et al., A microtransplantation approach for cell suspension grafting in the rat Parkinson model: a detailed account of the methodology. Neuroscience, 1994. 63(1): p. 57-72) in a way appropriate for injection of beads or Cultisphers of varying diameters from 50-200 μm (Malda, J., et al., Expansion of human nasal chondrocytes on macroporous microcarriers enhances redifferentiation. Biomaterials, 2003. 24(28): p. 5153-61; Voelker, C. C., et al., Selective neurofilament (SMI-32, FNP-7 and N200) expression in subpopulations of layer V pyramidal neurons in vivo and in vitro. Cereb Cortex, 2004. 14(11): p.1276-86; Huang, J., et al., A delivery strategy for rotenone microspheres in an animal model of Parkinson's disease. Biomaterials, 2005; and Calvo, P., et al., Quantification and localization of PEGylated polycyanoacrylate nanoparticles in brain and spinal cord during experimental allergic encephalomyelitis in the rat. Eur J Neurosci, 2002. 15(8): p. 1317-26). Initial experiments will not employ an immuno suppressive since it is expected that hASCs and Cutisphers will provide some protection from microglial invasion (to be determined by histology and immunostain for CD11b). However, if necessary, cyclosporine (i.p. 10 mg/kg, Novartis) will be administered daily beginning 3 days before surgery to evaluate transplant survival under immuno-supressive conditions (Klein, S. M., et al., GDNF delivery using human neural progenitor cells in a rat model of ALS. Hum Gene Ther, 2005.16(4): p.509-21).

Immunohistochemistry: Rats injected with a lethal dose of Beuthanasia Special (Schering Animal Health) will be perfused transcardially with chilled 0.9% saline and 4% paraformaldehyde/0.1M phosphate buffer. Tissue will be removed, post-fixed overnight, and transferred to 20% sucrose solution. The lumbar region will be cut in 50 μm sections on a cryostat. One in six sections will be processed for each of the antibodies: 1:100 antihuman nuclei (hnuclei, Chemicon, Temecula, Calif.), 1:250 antihuman GDNF (Chemicon), and anticholine acetyl transferase (1:100 ChAT, Chemicon). Additional sections will be stained for hASCs (Thy-1) and NSCs (nestin, Pax 6, both 1:100, Covance), anti-beta III tubulin (1:100, Covance), 1:200 (GFAP, Calbiochem), and immature neurons with Doublecortin (DCX, 1:1500, Abcam). Secondary antibodies used 1:1000 antimouse Alexa 488, 546 and 614 IgG1 (Molecular Probes, Eugene, Oreg.). Sections stained for anti-ChAT will be used for Quantitation in ventral horns. Generally, immunostains will be quantified by cell count, size, and immunointensity. Stained cells and sections will be imaged with a confocal microscope (Leica TCS SP2 or Perkin Elmer Ultraview) and quantified with Metamorph. Groups will be compared with ANOVA (p <0.05).

Specific Protocols

Standard culture of hESCs involves mouse feeders and DSR media containing bovine albumin. Humanized media including X-vivo 15, 20 (fully humanized defined culture media) and DMEM containing SSS supplement (general methods, above) is evaluated by culturing for 5 passages and maintenance of pluripotency is evaluated with cells grown on human feeders. Pluripotency is measured by (Oct-4+/nanog+/nestin−/GATA6−) immunostaining and PCR. Astrocytes are produced by plating hESC, line H7 or HSF6, on hASC at 50,000 cells/well and culturing for 19 days. NSCs are produced by plating on hASC at 300,000 to 600,000 cells/well and culturing in DSR for 19 days followed by N2 media+supplements (general methods, above). Motor neurons are produced from NSC (general methods, above). Motor neurons are distinguished by immunostaining and PCR for HB9, ChAT, and electrophysiological recordings. NSC are distinguished by (Pax6+/Sox1−) and nestin immunostaining. Astrocytes are distinguished by (GFAP+/nestin−/RC2−) immunostaining and PCR. HASC are identified by flow cytometry for (B-1 Integrin, CD 34, Integrin α4, Thy-1). Neu5Gc content of hESC is determined by anti Neu5Gc immunostain and flow cytometry (general methods).

Preliminary experiments have produced hESC-derived astrocytes on i-ASC co-culture at 50,000 cells/well. Further characterization and tests for motor neuron support by these astrocytes are pending. NSCs have been produced on MS-5 cells at 600,000 cells per well and derivation on hASCs at this density are in progress. Initial studies suggest a shift from astrocytes lineages to neuronal and oligodendrocytes lineages at higher density. The ability of hASCs to support NSC differentiation might depend on hASC source, patient age, or obesity. However, initial studies suggest that derivation of H7 hESCs into neuronal lineages is similar, independent of patient age (28-65) or obesity and further studies are in progress to evaluate the variability of hASC support of neuronal differentiation.

As an alternate to using human fibroblasts for hESC maintenance, Knock-out DMEM with FGF-2 and noggin (Xu, R. H., et al., Basic FGF and suppression of BMP signaling sustain undifferentiated proliferation of human ES cells. Nat Methods, 2005. 2(3): p. 185-90) can be evaluated. FGF-2 and noggin also show potential for differentiating hESC into NSC with N2B27 media and 8 ng/ml FGF and 100 ng/ml noggin on laminin substrate (Gerrard, L., L. Rodgers, and W. Cui, Differentiation of human embryonic stem cells to neural lineages in adherent culture by blocking BMP signaling. Stem Cells, 2005: p. 2005-0110). An alternative to using hASCs as feeder layers is to use human bone stromal cells (Cambrex), which can be cultured on X-vivo media. Alternates to HSF6 hESC available on site include H1, 7, 9, ES1, 2, 3 and BG01, 02 hESC. (H7 and ES03 are most suitable for NSC differentiation, Lorenz Studer, personal communication).

Transplanted cells may experience poor viability due to short-term trauma, poor adherence or failed integration after transplantation. Cultisphers provide mechanical support, promote survival, adhesion and neurite outgrowth (Bender, M. D., et al., Multi-channeled biodegradable polymer/CultiSpher composite nerve guides. Biomaterials, 2004. 25(7-8): p. 1269-1278) and have been effective in protecting neurosupportive cells for Parkinsons disease (Watts, R. L., et al., Stereotaxic intrastriatal implantation of human retinal pigment epithelial (hRPE) cells attached to gelatin microcarriers: a potential new cell therapy for Parkinson's disease. J Neural Transm Suppl, 2003(65): p. 215-27; Doudet, D. J., et al., PET imaging of implanted human retinal pigment epithelial cells in the MPTP-induced primate model of Parkinson's disease. Exp Neurol, 2004. 189(2): p. 361-8 and Bakay, R. A., et al., Implantation of Spheramine in advanced Parkinson's disease (PD). Front Biosci, 2004. 9: p. 592-602). Further, survival, differentiation, and functional integration might be improved with microencapsulated growth factors including FGF, CNTF (Tan, S. A., et al., Rescue of motoneurons from axotomy-induced cell death by polymer encapsulated cells genetically engineered to release CNTF. Cell Transplant, 1996. 5(5): p. 577-87) and GDNF (Fine, E. G., et al., GDNF and NGF released by synthetic guidance channels support sciatic nerve regeneration across a long gap. Eur J Neurosci, 2002. 15(4): p. 589-601).

Support cells (alternately, NSCs, hASCs, astrocytes) are seeded into Cultisphers by co-incubation in a suspension RCCS-D bioreactor (donated by Synthecon, Inc. Houston, Tex.). Microspheres that encapsulate FGF-2 have been previously prepared from PLGA (a FDA approved device made of a copolymer of poly(lactic acid) and poly(glycolic acid)), and similar techniques have been successful for proteins as large as BSA (Meese, T. M., et al., Surface studies of coated polymer microspheres and protein release from tissue-engineered scaffolds. J Biomater Sci Polym Ed, 2002. 13(2): p. 141-51; Hu, Y., J. O. Hollinger, and K. G. Marra, Controlled release from coated polymer microparticles embedded in tissue-engineered scaffolds. J Drug Target, 2001. 9(6): p. 431-8 and Royce, S. M., M. Askari, and K. G. Marra, Incorporation of polymer microspheres within fibrin scaffolds for the controlled delivery of FGF-1. J Biomater Sci Polym Ed, 2004. 15(10): p. 1327-36). Established protocols are used for encapsulation of CNTF and GDNF in an emulsion of 100 um, controlled by surfactant.

We have evaluated the Synthecon bioreactor for support of hASC culture on microcarrier beads, and find that hASCs in suspension attach to microcarrier beads. It is anticipated from published work (Watts, R. L., et al., Stereotaxic intrastriatal implantation of human retinal pigment epithelial (hRPE) cells attached to gelatin microcarriers: a potential new cell therapy for Parkinson's disease. J Neural Transm Suppl, 2003(65): p. 215-27; Doudet, D. J., et al., PET imaging of implanted human retinal pigment epithelial cells in the MPTP-induced primate model of Parkinson's disease. Exp Neurol, 2004. 189(2): p. 361-8; Bakay, R. A., et al., Implantation of Spheramine in advanced Parkinson's disease (PD). Front Biosci, 2004. 9: p. 592-602 and Bender, M. D., et al., Multi-channeled biodegradable polymer/CultiSpher composite nerve guides. Biomaterials, 2004. 25(7-8): p. 1269-1278), that similar methods will be successful for seeding Cultisphers with support stem cell and products. Three-dimensional neural guides have also been successfully seeded with HASC by co-culture and similar techniques will be applied for seeding NSCs, astrocytes and motor neurons. Optimization of cell density, tests of differentiation fate and viability can be tested. In the use of growth factors, such as FGF-2, slow release of such factors for 1-3 weeks in solution (FIG. 8) is anticipated, longer when enclosed in tissue, sufficient to guide differentiation and integration of transplanted cells, for example and without limitation, into the spinal cord microenvironment.

Cultisphers of standard size range from 150-400 μm. Current transplantation technology employs injection needles up to 100 μm in diameter (Klein, S. M., et al., GDNF delivery using human neural progenitor cells in a rat model of ALS. Hum Gene Ther, 2005. 16(4): p. 509-21). The injection needle can be varied in size up to 200 μm to facilitate transplantation of larger sizes. If standard size carrier beads are too large for transplantation by modified needle injection, the preparation can be morselized by mechanical means and subsequent sieving to produce beads of defined sizes as small as 50 um. It is possible that there is a minimum bead size due, not only to mechanical considerations, but also potentially because of a critical minimum number of cells per colony necessary for survival or differentiation. Microspheres size also can be controlled by fabrication conditions such as stirring rate and surfactant concentration. Growth factor release curves can be evaluated as a function of bead size.

Differentiation in vivo from NSCs is likely to promote better functional integration. In addition, NSCs and MSCs may promote immunotolerance. However, beyond assays of inflammation within the graft, immunotolerance can be evaluated with cell-based tests now in development. The differentiation of NSCs and the effects of microspheres on the fate of transplanted cells and rat motor neurons can be documented.

Initially, the effects of density of NSCs, hASCs and astrocytes on hESC-derived motor neuron survival and neurite regrowth can be tested after axotomy at 2 weeks on laminin or collagen substrates in basal N2 media (general methods). Subsequently, support cells, alone and in combination can be seeded in Cultisphers and evaluated for their ability to promote motor neuron survival, neurite length, and electrophysiological characteristics at 4 weeks. These conditions can be compared to FF2 human foreskin fibroblasts or empty Cultisphers as controls. Following optimization of support cell density for 2 weeks, seeded Cultisphers can be cultured with adherent motor neurons for 30-60 days and cultures can be evaluated by serial confocal microscopy for elaborated motor neuron neural networks and support cell survival and differentiation with antibodies against markers (ChAT, HB9, VAChT), structural proteins (beta III tubulin) and pre (synaptophysin) and post (PSD95) markers.

While animal studies are important for determining safety and efficacy of support cells, the mechanism of action, and the necessity and sufficiency of each support cell lineage should first be determined in vitro. Additionally, support cell density and age as well as growth factor and matrix conditions can be optimized more efficiently in vitro. The in vitro tests help determine whether support cells are directly or indirectly effective in subsequent in vivo tests. WT hESC-derived motor neurons survival should be improved by the addition of supportive cells since basal conditions and necessary growth factors at each stage of derivation have been defined. If support of mature neurons does not produce an improvement in motor neuron viability, outgrowth or electrophysiology, the ability of support cells to contribute to the final stages of motor neuron differentiation from NSC or to the ability of WT or SOD mutant motor neurons to resist oxidative challenge can be evaluated as outlined below. Astrocytes vary in their ability to support neurons, for example hippocampal but not spinal cord astrocytes are supportive of neuron injury (Song, H., C. F. Stevens, and F. H. Gage, Astroglia induce neurogenesis from adult neural stem cells. Nature, 2002. 417(6884): p.39-44). We have chosen 19 days for astrocytes preparation because we believe slightly less mature astrocytes are more likely to adapt to local environments, but if these astrocytes are not supportive, more mature astrocytes from 30-day cultures can be evaluated. hASC support differentiation and growth of neurons in vitro and in the ability of hASC to differentiate hESC into motor neurons with appropriate basal media and growth factor sequence (general methods) can be tested. It is expected that motor neurons would be supported as well. NSCs are neurosupportive and have the potential to respond to the injury environment by differentiation into supportive cells (Llado, J., et al., Neural stem cells protect against glutamate-induced excitotoxicity and promote survival of injured motor neurons through the secretion of neurotrophic factors. Mol Cell Neurosci, 2004. 27(3): p.322-31). The use of NSCs as support cells is more speculative than the use of astrocytes or hASCs as support cells and NSCs might not persist but might differentiate in a way dependent on the microenvironment.

As an alternative to WT hESC-derived motor neurons, we could overexpress SODI cells by transfecting NSC with human wild type (wt) or mutant (G93A) SOD1 (Rizzardini, M., et al., Low levels of ALS-linked Cu/Zn superoxide dismutase increase the production of reactive oxygen species and cause mitochondrial damage and death in motor neuron-like cells. J Neurol Sci, 2005. 232(1-2): p. 95-103) and subsequent differentiate into motor neurons. (G93A) SOD1 neurons in vitro show toxicity at plateau phase growth, and sensitivity to oxidative stress by depletion of glutathione by ethacrynic acid and mitochondrial dysfunction induced by rotenone-mediated inhibition of complex I (Rizzardini, M., et al., Low levels of ALS-linked Cu/Zn superoxide dismutase increase the production of reactive oxygen species and cause mitochondrial damage and death in motor neuron-like cells. J Neurol Sci, 2005. 232(1-2): p. 95-103). HESC derived from ALS patients by nuclear transfer of patient fibroblasts into oocytes are available. When prepared, these cells would allow in vitro testing of support cells that guard ALS patient-derived motor neurons from oxidative stress. As an alternate to neural guides, collagenous cell support matrices (Cultisphers) or isometric matrices could be evaluated. Neural stem cell derivation from hESC on synthetic matrices using poly (lactic-co-glycolic acid) and poly (L-lactic acid) has been shown to produce neural rosette-like structures when differentiation agents are included in the medium (Levenberg, S., et al., Neurotrophin-induced differentiation of human embryonic stem cells on three-dimensional polymeric scaffolds. Tissue Eng, 2005. 11(3-4): p. 506-12). In vitro, media addition of growth factors is a baseline approach and is sufficient for proof of principle.

In vivo Studies:

Transgenic human astrocytes that secrete GDNF, which are transplanted into the lumbar spinal cord of rats overexpressing the G93A SOD I mutation are locally neuroprotective (Klein, S. M., et al., GDNF delivery using human neural progenitor cells in a rat model of ALS. Hum Gene Ther, 2005. 16(4): p. 509-21) and clinical trials are currently in progress (ALS web site). In addition, Cultisphers have been used successfully to implant dopamine secreting support cell in Parkinson's animal models and in clinical trials, showing survival of implants, immunosuppression, secretion of diffusible, neuroprotective dopamine, and clinical improvement. These results suggest that cell therapy using neuron support systems are feasible and that the long-term survival of allogenic cells that secrete conditioning agents might provide patients with a delivery system that is effective against neurodegenerative disease. The ability of transplanted cells to survive transplantation into WT animals can be tested first. The WT animal injections can take place along side the in vitro tests so that experimental results will provide feed back for optimization of cell preparation and transplant viability. After optimization, and determination of viability and safety in vivo, the efficacy in SOD I mutant rats can then be tested.

It is expected that optimized cell preparations and growth factors will include hASCs and astrocytes in Cultisphers (Condition 1), NSCs and hASCs in Cultisphers (condition 2) and microencapsulated GDNF (condition 3). First, we can evaluate injection of Cultisphers alone vs. Condition 1 and PLGA microspheres with 0, 10 and 50 ng/ml GDNF (5 conditions X 4 animals X 3 replicates=60 WT animals. Then, we can evaluate injection of control fibroblasts FF2 in Cultisphers (Condition 6), HASC+NSC Cultisphers, +GDNF microspheres (Condition 7), and HASC+NSC Cultisphers+astrocytes Cultisphers+GDNF microspheres (Condition 8). (3 conditions×4 animals×3 replicates=36 animals). It is believed that the effects of injected astrocytes+GDNF will be greatest, but NSCs+hASCs offer potential for better adaptability to local injury and oxidative damage caused by ALS-like environment of the SOD1 mutant. There is also the possibility of motor neuron replacement. It is anticipated that GDNF will provide short-term support of endogenous motor neurons and astrocytes and importantly will promote survival and integration of the graft. WT astrocytes will provide longer term conditioning of the local environment in the spinal cord.

Later, the optimal Condition (from 1-8 evaluated above), can be employed in SOD1 mutant rats and compared to cell-free beads and growth factor-free microcarriers. Symptoms appear in these animals at approximately 3 months and death occurs at 4 months. Transplantation can take place at 2 months and histological evaluation can be made 6 weeks later. Behavioral modifications of animals (weight, hind quarter locomotion) can be evaluated before sacrifice of control and experimental animals.

Primary alternates are included within the experimental section and described as conditions 1-8. If the effects of optimized condition chosen from WT rat experiments do not have measurable effects in SODI mutant animals, then the next optimal condition will be chosen for evaluation.

Having described this invention, it will be understood to those of ordinary skill in the art that the same can be performed within a wide and equivalent range of conditions, formulations and other parameters without affecting the scope of the invention or any embodiment thereof.

We claim:

1. A method of culturing a successor cell from a precursor cell comprising co-culturing a precursor cell with an adult mesenchymal stem cell.

2. The method of claim 1, wherein the adult mesenchymal stem cell is derived from adipose tissue.

3. The method of claim 1, wherein the successor cell is one of an astrocyte, an oligodendrocyte and a neuron.

4. The method of claim 1, further comprising co-culturing the precursor cell and the adult mesenchymal stem cell on a growth matrix biologically-compatible with the cells.

5. The method of claim 4, wherein the growth matrix comprises collagen.

6. The method of claim 5, wherein the growth matrix further comprises a hydrogel.

7. The method of claim 6, wherein the hydrogel comprises one or more of poly(lactic-co-glycolic acid) and polycaprolactone.

8. The method of claim 6, wherein the hydrogel comprises laminin.

9. The method of claim 4, wherein the growth matrix comprises a polymer.

10. The method of claim 9, wherein the polymer is a hydrogel.

11. The method of claim 9, wherein the polymer is biodegradable.

12. The method of claim 9, wherein the polymer comprises one or more of a polyesters, polysaccharides, poly(lactic acid), poly(glycolic acid), poly(lactic-co-glycolic acid) copolymers, poly(vinyl alcohol), polycaprolactone, polyethyleneglycol and gelatin.

13. The method of claim 9, wherein the polymer comprises a copolymer.

14. The method of claim 13, wherein the copolymer is poly(lactic-co-glycolic acid).

15. The method of claim 2, wherein the growth matrix comprises one or more of a growth factor, a differentiation factor and a migration factor.

16. The method of claim 15, wherein the growth matrix comprises a neurotropic fractor.

17. The method of claim 16, wherein the neurotropic factor is one or more of glial derived neurotrophic factor, cilliary neurotrophic factor and fibroblast growth factor 2.

18. The method of claim 15, wherein the growth factor, a differentiation factor and a migration factor is associated with a hydrogel matrix.

19. The method of claim 18, wherein the hydrogel matrix comprises poly(lactic-co-glycolic acid).

20. The method of claim 4, wherein the growth matrix comprises a polypeptide comprising one or more of the following amino acid sequences: RGD, YIGSR (SEQ ID NO: 1), IKVAV (SEQ ID NO: 2) and GRGDS (SEQ ID NO: 3).

21. The method of claim 1, wherein the precursor cell and the adult mesenchymal stem cell are obtained from the same patient.

22. The method of claim 1, wherein the precursor cell and the adult mesenchymal stem cell are obtained from the same human patient.

23. The method of claim 1, wherein the precursor cell is an embryonic stem cell and the adult mesenchymal stem cell is human.

24. The method of claim 1, wherein the precursor cell is an embryonic stem cell.

25. The method of claim 1, wherein the precursor cell is a neuronal stem cell.

26. The method of claim 1, further comprising modulating density of the adult mesenchymal stem cell to determine successor cell type.

27. A composition comprising mesenchymal stem cells in a growth matrix biologically-compatible with the cells.

28. The composition of claim 27, wherein the growth matrix comprises collagen.

29. The composition of claim 28, wherein the growth matrix further comprises a hydrogel.

30. The composition of claim 29, wherein the hydrogel comprises one or more of poly(lactic-co-glycolic acid) and polycaprolactone.

31. The composition of claim 29, wherein the hydrogel comprises laminin.

32. The composition of claim 27, wherein the growth matrix comprises a polymer.

33. The composition of claim 32, wherein the polymer is a hydrogel.

34. The composition of claim 32, wherein the polymer is biodegradable.

35. The composition of claim 32, wherein the polymer comprises one or more of a polyesters, polysaccharides, poly(lactic acid), poly(glycolic acid), poly(lactic-co-glycolic acid) copolymers, poly(vinyl alcohol), polycaprolactone, polyethyleneglycol and gelatin.

36. The composition of claim 32, wherein the polymer comprises a copolymer.

37. The composition of claim 36, wherein the copolymer is poly(lactic-co-glycolic acid).

38. The composition of claim 27, wherein the growth matrix comprises one or more of a growth factor, a differentiation factor and a migration factor.

39. The composition of claim 38, wherein the growth matrix comprises a neurotropic fractor.

40. The composition of claim 39, wherein the neurotropic factor is one or more of glial derived neurotrophic factor, cilliary neurotrophic factor and fibroblast growth factor 2.

41. The composition of claim 38, wherein the growth factor, a differentiation factor and a migration factor is associated with a hydrogel matrix.

42. The composition of claim 40, wherein the hydrogel matrix comprises poly(lactic-co-glycolic acid).

43. The composition of claim 27, wherein the growth matrix comprises a polypeptide comprising one or more of the following amino acid sequences: RGD, YIGSR (SEQ ID NO: 1), IKVAV (SEQ ID NO: 2) and GRGDS (SEQ ID NO: 3).

44. The composition of claim 27, wherein the adult mesenchymal stem cell is derived from adipose tissue.

45. The composition of claim 27, wherein the successor cell is one of an astrocyte, an oligodendrocyte and a neuron.

46. The composition of claim 27, wherein the precursor cell and the adult mesenchymal stem cell are obtained from the same patient.

47. The composition of claim 27, wherein the precursor cell and the adult mesenchymal stem cell are obtained from the same human patient.

48. The composition of claim 27, wherein the precursor cell is an embryonic stem cell.

49. A method of regenerating tissue comprising introducing into a patient a cell growth niche comprising mesenchymal stem cells in a growth matrix biologically-compatible with the cells.

50. The method of claim 49, wherein the growth matrix comprises collagen.

51. The method of claim 50, wherein the growth matrix further comprises a hydrogel.

52. The method of claim 51, wherein the hydrogel comprises one or more of poly(lactic-co-glycolic acid) and polycaprolactone.

53. The method of claim 51, wherein the hydrogel comprises laminin.

54. The method of claim 49, wherein the growth matrix comprises a polymer.

55. The method of claim 53, wherein the polymer is a hydrogel.

56. The method of claim 53, wherein the polymer is biodegradable.

57. The method of claim 53, wherein the polymer comprises one or more of a polyesters, polysaccharides, poly(lactic acid), poly(glycolic acid), poly(lactic-co-glycolic acid) copolymers, poly(vinyl alcohol), polycaprolactone, polyethyleneglycol and gelatin.

58. The method of claim 54, wherein the polymer comprises a copolymer.

59. The method of claim 58, wherein the copolymer is poly(lactic-co-glycolic acid).

60. The method of claim 49, wherein the growth matrix comprises one or more of a growth factor, a differentiation factor and a migration factor.

61. The method of claim 60, wherein the growth matrix comprises a neurotropic factor.

62. The method of claim 61, wherein the neurotropic factor is is one or more of glial derived neurotrophic factor, cilliary neurotrophic factor and fibroblast growth factor 2.

63. The method of claim 60, wherein the growth factor, a differentiation factor, an adhesion factor and a migration factor is associated with a hydrogel matrix.

64. The method of claim 63, wherein the hydrogel matrix comprises poly(lactic-co-glycolic acid).

65. The method of claim 49, wherein the growth matrix comprises a polypeptide comprising one or more of the following amino acid sequences: RGD, YIGSR (SEQ ID NO: 1), IKVAV (SEQ ID NO: 2) and GRGDS (SEQ ID NO: 3).

66. The method of claim 49, wherein the mesenchymal stem cells is derived from adipose tissue.

67. The method of claim 49, wherein the successor cell is one of an astrocyte, an oligodendrocyte and a neuron.

68. The method of claim 49, wherein the precursor cell and the mesenchymal stem cells are obtained from the same patient.

69. The method of claim 49, wherein the precursor cell and the mesenchymal stem cells are obtained from the same human patient.

70. The method of claim 49, wherein the precursor cell is an embryonic stem cell.

71. The method of claim 49, wherein the precursor cell is a neuronal stem cell.

72. The method of claim 49, further comprising modulating density of the mesenchymal stem cells in order to control cell differentiation in the niche.

73. A method of repairing damage to a patient's nervous system comprising transplanting into the patient's nervous system a composition comprising mesenchymal stem cells in a growth matrix biologically-compatible with the cells.

74. The method of claim 73, wherein the growth matrix comprises collagen.

75. The method of claim 73, wherein the growth matrix further comprises one or more of a growth factor, a differentiation factor and a migration factor.

76. The method of claim 73, wherein the growth matrix comprises a neurotropic factor.

77. The method of claim 76, wherein the neurotropic factor is one or more of glial derived neurotrophic factor, cilliary neurotrophic factor and fibroblast growth factor 2.

78. The method of claim 73, wherein the growth matrix comprises a hydrogel.

79. The method of claim 73, wherein the patient has amyotrophic lateral sclerosis.


SEQUENCE LISTING (3 sequences)

SEQ ID NO: 1 - protein, 5 aa, Homo sapiens
YIGSR

SEQ ID NO: 2 - protein, 5 aa, Homo sapiens
IKVAV

SEQ ID NO: 3 - protein, 5 aa, Homo sapiens
GRGDS